(19) United States
(12) Patent Application Publication
(10) Pub. No.: US 2013/0060259 A1
(43) Pub. Date: Mar. 7, 2013

(54) VACUUM DELIVERY EXTRACTOR

(71) Applicant: Association for Public Health, Jerusalem (IL)

(72) Inventor: Gadi PORAT, Jerusalem (IL)

(73) Assignee: ASSOCIATION FOR PUBLIC HEALTH, Jerusalem (IL)

(21) Appl. No.: 13/659,989

(22) Filed: Oct. 25, 2012

Related U.S. Application Data
(63) Continuation-in-part of application No. 12/435,832, filed on May 5, 2009.
Application No. 12/435,832 is a continuation-in-part of application No. PCT/IL2008/001442, filed on Nov. 3, 2008.
(60) Provisional application No. 60/996,254, filed on Nov. 8, 2007.

(51) Int. Cl. A61B 17/42

(52) U.S. Cl. 606/123

(57) ABSTRACT

A delivery extractor for use with a suction source and at least one monitor in vacuum assisted deliveries. The extractor comprises a cup-shaped element and a tubular stem joined to the cup-shaped element and in pneumatic communication with the suction source. The extractor also includes at least one noninvasive sensor positioned on a sensor support so as to continuously contact the scalp of a fetus when the head of the fetus is positioned in the cup-shaped element. The sensor support is depressible within the cup-shaped element. The at least one sensor is in communication with the monitor(s) which monitors at least one physiological indicator of the well-being of the fetus during its transit through the birth canal. In other cases, the aforementioned noninvasive sensor(s) detects direct waves while other noninvasive sensors positioned on the rim of the cup-shaped element detect surface waves related to the same phenomenon.

### CROSS REFERENCE TO RELATED APPLICATIONS

The present application is a continuation-in-part application of U.S. application Ser. No. 12/435,832, filed May 5, 2009, which itself is a national phase and continuation-in-part application of international application PCT/IL2008/001442, filed Nov. 3, 2008, which itself claims priority from U.S. Provisional Application Ser. No. 60/996,254, filed Nov. 8, 2007, entitled “VACUUM DELIVERY EXTRACTOR”, the disclosure of which is incorporated herein by reference.

### FIELD OF THE INVENTION

The present invention relates to a vacuum delivery extractor which allows for monitoring of a fetus as it transits the birth canal during a vacuum assisted delivery.

### BACKGROUND OF THE INVENTION

At present, about eight percent of births in the United States are vacuum assisted deliveries. Fetal and maternal indications which generally lead to vacuum assisted deliveries include a non-reassuring fetal heart tracing, a prolonged second stage of labor and failure to progress in the second stage of labor. This procedure, while generally safe, does at times lead to long-term medical problems for the neonate. When suction is not monitored accurately, brain or other damage to the fetus may occur. In order to prevent such occurrences accurate monitoring of the fetus during the final stages of labor and delivery is needed.

At present, the quality of monitoring the heart rate of a fetus transiting through the birth canal is generally poor. Often, external ultrasound instrumentation is used with the ultrasound device placed on the mother's stomach. However, differentiating between the fetal heart rate from the mother's heart rate, the mother's labor contractions, and changes in the mother's and/or the fetus's overall position is not easy using ultrasound devices. Moreover, an obese patient makes monitoring the fetus's condition using ultrasound difficult.

Internal fetal scalp electrodes (FSE), generally in the form of a sensor sheathed in a plastic catheter, have also been used to measure fetal heart rate prior to the fetus's entering the birth canal. Such electrodes, long well-known in the art, are inserted through the mother's vagina, past her cervix, until the tip of the electrode touches the scalp of the fetus and is held there. Scalp electrodes almost always result in piercing the scalp's epidermis, making trauma, particularly scalp hematomas which can lead to infection, including meningitis, and intracranial hemorrhaging possible. Many of these conditions require treatment of the neonate after birth. Additionally, fetal scalp electrodes restrict the mother's movement during labor often causing discomfort, and even unnecessary pain, to the mother. Fetal scalp electrodes are not used while the fetus is passing through the birth canal, especially during vacuum assisted deliveries where a vacuum delivery extractor must be positioned on the head of the fetus. When FSEs are used they generally measure pulse or heart rate and they usually are not used for making electrocardiogram (ECG) measurements.

Today, there is no fully adequate non-invasive method to track fetal heart or pulse rate or to provide a full ECG during the last stages of a vacuum assisted delivery, that is, while the fetus is transiting the birth canal.

Generally, when a fetus is passing through the birth canal during vacuum assisted deliveries, the vacuum system, and not the fetus, is monitored. Typically, the magnitude and duration of the vacuum suction pulses being applied by the vacuum delivery extractor system to the fetus is what is tracked. It is known that prolong exposure to the highest vacuum required for the delivery, typically, approximately 600 mm Hg, may leave the fetus permanently disabled. In addition, if vacuum suction is not controlled and it exceeds a maximum allowed value as defined in medically tested and approved protocols, the neonate may emerge into the world permanently disabled.

In view of the above, it would be advantageous to develop a device for monitoring the condition of the fetus in the last stages of a vacuum assisted delivery, i.e. passage through the birth canal. The device should preferably be non-invasive to the fetus. It would also be advantageous to develop a device that is inexpensive and suitable for one-time use.

### SUMMARY OF THE PRESENT INVENTION

It is an object of the present invention to provide a vacuum delivery extractor, also at times herein denoted as an extractor or a delivery extractor or a vacuum assisted delivery extractor, that allows for tracking of the fetus's heart rate and/or pulse rate and/or ECG and/or blood oxygen level and/or other physiological indicator of the well-being of the fetus while the fetus is transiting the birth canal during the last stages of delivery.

It is another object of the present invention to provide a vacuum delivery extractor that allows for readings that do not require invasion of the scalp of the fetus when continuously monitoring fetal heart rate (FHR), fetal pulse rate (FPR), fetal electrocardiogram (FECG), or fetal oxygen saturation levels (FSpO₂). The extractor may be adaptable to measure other physiological indicators of the condition of the fetus.

It is another object of the present invention to provide an inexpensive vacuum delivery extractor which also monitor's the fetus's condition and which is suitable for one-time use.

It is an object of the present invention to provide a vacuum delivery extractor system that allows for tracking of the fetus's heart rate and/or pulse rate and/or ECG and/or oximetry reading while the fetus is transiting the birth canal during the last stages of delivery.

It is a further object of the present invention to provide a method for vacuum assisted deliveries where the fetus's well being can be tracked continuously as it transits the birth canal.

In one aspect of the present invention there is provided a delivery extractor for use with a vacuum suction source and one or more monitors in a vacuum assisted delivery. The extractor comprises a cup-shaped element and a tubular stem having a first end and a second end, the first end joined to the cup-shaped element and the second end in pneumatic communication with the vacuum suction source. The extractor also includes one or more resilient sensors positioned on a sensor support. The support is compressible within the cup-shape element and movable therein substantially in the direction of the tubular stem. The one or more sensors are situated generally in the center of the cup-shaped element so as to be in continuous contact with the scalp of a fetus when the cup-shaped element is positioned on the head of the fetus and the vacuum suction source is activated. The one or more sensors are in communication with the one or more monitors which monitor one or more physiological indicators of the well-being of the fetus during the transit of the fetus through the birth canal during a vacuum assisted delivery.

In an embodiment of the extractor, the extractor further includes a sensor housing which is recessed in the sensor support. The one or more sensors are positioned in the sensor housing and protrude therefrom and the extractor includes a spring which is in operative connection with the sensor support, the spring allowing the sensor support to be compressed within the cup-shaped element.

In still another embodiment of the extractor, the sensor support is formed of a resilient material allowing said support to be compressed within said cup-shaped element.

In yet another embodiment of the extractor, the one or more sensors are sensors that measure one or more of the following physiological indicators of the well being of the fetus: fetal heart rate (FHR), fetal pulse rate (FPR), fetal ECG, and blood oxygen levels (oximetry). When multiple measurements or multiple types of measurements are made, they may be made concurrently.

In some embodiments of the extractor, the one or more sensors are electrically conductive sensors in electrical communication with a set of isolated wires which conduct electrical signals generated by the one or more sensors when the sensors contact the scalp of the fetus. They measure one or more physiological indicators of the well-being of the fetus. In some instances, the isolated wires extend from the one or more sensors through the hollow of the tubular stem, exiting from the stem through a hermetically sealed join; they then are joined and are in electrical communication with the one or more monitors. In other instances, the isolated wires conduct electrical signals to a wireless transmitter, the wireless transmitter transmitting signals to a wireless receiver in communication with the one or more monitors. The wireless transmitter may be positioned at a location selected from the following group of locations: within the stem of the extractor and within a grip, the grip located on the stem and operative for holding the extractor while positioning the cup.

In yet another aspect of the present invention, there is provided a system for monitoring the condition of a fetus during the later stages of a vacuum assisted delivery. The system comprises a delivery extractor, a vacuum suction source and one or more monitors. The delivery extractor comprises a cup-shaped element and a tubular stem having a first end and a second end, the first end being joined to the cup. The delivery extractor also includes one or more non-invasive resilient sensors positioned on a sensor support. The support is compressible within the cup-shape element and movable therein substantially in the direction of the tubular stem. The one or more sensors are situated generally in the center of the cup-shaped element so as to be in continuous contact with the scalp of a fetus when the cup-shaped element is positioned on the head of the fetus and the vacuum suction source is activated. The vacuum suction source is removably connectable with the second end of the stem and is in pneumatic communication with the cup-shaped element. The one or more monitors are in communication with the one or more sensors and receive signals therefrom. The one or more monitors monitor one or more physiological indicators of the well-being of the fetus during the transit of the fetus through the birth canal during the later stages of a vacuum assisted delivery.

In an embodiment of the system, the extractor further includes a sensor housing which is recessed in the sensor support. The one or more sensors are positioned in the sensor housing and protrude therefrom and the extractor includes a spring which is in operative connection with the sensor support, the spring allowing the sensor support to be compressed within the cup-shape element.

In still another embodiment of the system, the sensor support of the extractor is formed of a resilient material allowing said support to be compressed within said cup-shaped element.

In yet another embodiment of the system, the stem is equipped with an emergency suction reduction means which can be activated to interrupt suction produced by the vacuum suction source. This activation occurs when the one or more sensors indicate that the fetus may be in distress or when a physician has otherwise determined that the vacuum delivery procedure must be aborted.

In a further embodiment of the system, the stem is equipped with a vacuum modulating means operative to modulate the vacuum produced by the vacuum suction source when a physician has determined that the vacuum strength is to be increased or decreased during the delivery.

In still another embodiment, the one or more sensors are sensors that measure one or more of the following physiological indicators of the well being of the fetus: fetal heart rate (FHR), fetal pulse rate (FPR), fetal ECG, and fetal blood oxygen levels (oximetry).

In some instances, the one or more monitors are adapted to receive and interpret signals from the one or more types of sensors. When multiple measurements or multiple types of measurements are made, they may be made concurrently.

In another embodiment of the system, the system further includes a wireless transmitter in communication with the one or more sensors. The transmitter receives electrical signals from the sensors so as to transmit signals to a wireless receiver in electrical communication with the one or more monitors.

In yet another embodiment of the system, the one or more monitors further include a controller that disconnects the vacuum suction source when the controller detects that the measured physiological indicator of well being is greater or less than predetermined maximum and minimum values for that indicator.

In still another embodiment of the system, the vacuum suction source includes a controller which is operative to disconnect the vacuum suction source when the suction produced by the source exceeds a predetermined value for the maximum suction to be applied or when the total duration during which the maximum suction has been applied is greater than a predetermined value for the total duration for which maximum suction is to be applied.

In some instances the one or more monitors activate a warning device when the controller determines that the measured physiological indicator of well-being is above or below predefined limits.

In another aspect of the present invention there is provided a method for monitoring the well-being of a fetus during a vacuum assisted delivery. The second method comprises the steps of:
placing a vacuum assisted delivery extractor on the head of a fetus as it enters the birth canal, thereby to position against the scalp of the fetus at least one non-invasive monitoring sensor for monitoring one or more physiological indicators of the well-being of the fetus;
activating a vacuum suction source during the mother's contractions for assisting movement of the fetus along the birth canal and to hold the monitoring sensor in sensory contact with the scalp of the fetus; and
monitoring the one or more physiological indicators of the well-being of the fetus via the at least one non-invasive monitoring sensor as the fetus moves along the birth canal.

In an embodiment of the method, the step of activating includes allowing the suction to reach a first value during the mother's contractions and then allowing the suction to reach a second value between the mother's contractions, where the second value is less than the first value.

In another embodiment of the method, the step of activating includes progressively increasing the vacuum suction until a predetermined maximum value for the vacuum suction is attained and then maintaining that value until the mandible of the fetus passes the mother's pubic symphysis or until the physician otherwise aborts the vacuum assisted delivery.

In yet another embodiment of the method of the present invention, the method further includes the step of aborting the step of activating a suction source when one or more of the following conditions is indicated: the step of monitoring indicates that the fetus is in distress; the step of monitoring indicates that the maximum suction being applied exceeds a predetermined value for the maximum suction to be applied; and the step of monitoring indicates that the total duration for which the maximum suction has been applied exceeds a predetermined value.

In still another embodiment of the method, the method further includes the step of modulating the strength of the vacuum produced by the vacuum suction source when at least one of the following conditions is indicated: the step of monitoring indicates that the fetus is in distress; the step of monitoring indicates that the maximum suction being applied exceeds a predetermined value for the maximum suction to be applied; the step of monitoring indicates that the total duration at which the maximum suction has been applied exceeds a predetermined value for the maximum duration; and the physician has determined that the vacuum suction strength is to be increased or decreased.

In an aspect of the present invention, there is provided a delivery extractor for use in a vacuum assisted delivery with a vacuum suction source and one or more monitors for monitoring one or more physiological indicators of the well being of a fetus. The extractor includes: a cup-shaped element having a rim; a tubular stem having a first end and a second end, the first end joined to the cup-shaped element and the second end in pneumatic communication with the vacuum suction source; and one or more noninvasive first sensors supported within a sensor support, the support depressible within the cup-shaped element and movable therein toward the tubular stem, and one or more noninvasive second sensors positioned on the rim of the cup-shaped element. The one or more first sensors are operative to detect electrical signals of direct waves arising from an electrophysiological event and the one or more second sensors are operative to detect electrical signals of surface waves arising from the electrophysiological event.

It should be noted that as used herein, direct waves refer to waves that travel through the fetus from their point of origin to and through the skull of the fetus to the one or more first sensor. Surface waves travel through the fetus from their point of origin, exiting the fetus, and traveling through a wet environment surrounding the fetus to the one or more second sensors.

In one embodiment of this aspect of the delivery extractor, the one or more first sensors are situated generally in the center of the cup-shaped element so as to be in continuous contact with the scalp of a fetus when the head of the fetus is fully engaged with the cup-shaped element, and wherein the one or more first and second sensors are in electrical communication with the one or more monitors.

In some embodiments of the delivery extractor, the one or more second sensor is a plurality of second sensors positioned along the rim of the cup-shaped element.

In yet other embodiments of the delivery extractor, the one or more second sensors are positioned on a flexible support adjacent to the rim of the cup-shaped element and depressible therein.

In still other embodiments of the delivery extractor, the extractor further includes a spring and a sensor housing, the sensor housing being recessed in the sensor support with the one or more first sensors positioned in the sensor housing and protruding therefrom, and the sensor support being in operative connection with the spring so that the sensor support may be depressed within the cup-shaped element.

In embodiments of the delivery extractor, the one or more first sensors and the one or more second sensors monitor one or more of the following physiological indicators of the well being of the fetus: fetal heart rate (FHR), fetal pulse rate (FPR), and fetal ECG.

In yet another embodiment of the delivery extractor, the first and second sensors are in electrical communication with a wireless transmitter. The wireless transmitter transmits signals to a wireless receiver in electrical communication with the one or more monitors.

In yet another embodiment of the delivery extractor, the sensor support has a conductive plate positioned thereon, the plate having a tube integrally formed therewith extending substantially transversally from the plate and the one or more first sensor is configured and sized to be positioned and held within the tube.

In another aspect of the present invention, there is provided a system for monitoring the condition of a fetus during a vacuum assisted delivery, the system including: a delivery extractor; a vacuum suction source removably connectable with the second end of a tubular stem and in pneumatic communication with a cup-shaped element; and one or more monitors, in communication with the one or more first sensors and the one or more second sensors, receiving signals from the sensors. The one or more monitors monitor one or more physiological indicators of the well-being of the fetus. The delivery extractor includes: a cup-shaped element having a rim; a tubular stem having a first end and a second end, the first end joined to the cup-shaped element; and one or more noninvasive first sensors supported within a sensor support, the support depressible within the cup-shaped element and movable therein toward the tubular stem, and one or more noninvasive second sensors positioned on the rim of the cup-shaped element. The one or more first sensors are operative to detect electrical signals of direct waves arising from an electrophysiological event and the one or more second sensors are operative to detect electrical signals of surface waves arising from the electrophysiological event;

In an embodiment of the system, the one or more first sensors are situated generally in the center of the cup-shaped element so as to be in continuous contact with the scalp of a fetus when the head of the fetus is fully engaged with the cup-shaped element. The one or more first sensors and the one or more second sensors are in electrical communication with the one or more monitors.

In yet another embodiment of the system, the system further includes a spring and a sensor housing, the sensor housing being recessed in the sensor support. The one or more first sensors are positioned in the sensor housing and protrude therefrom. The sensor support is in operative connection with the spring so that the sensor support may be depressed within the cup-shaped element.

In still another embodiment of the system, the stem is equipped with an emergency suction reduction means which can be activated to interrupt the suction produced by the vacuum suction source when the one or more first sensors and the one or more second sensors indicate that the fetus may be in distress or when a physician has otherwise determined that the vacuum delivery procedure must be aborted.

In a further embodiment of the system, the stem is equipped with a vacuum modulating means operative to modulate the vacuum produced by the vacuum suction source when a physician has determined that the vacuum strength is to be increased or decreased during the delivery.

In yet another embodiment of the system, the system further includes a wireless transmitter in electrical communication with the one or more first sensors and the one or more second sensors receiving electrical signals therefrom so as to transmit signals to a wireless receiver in electrical communication with the one or more monitors.

In still another embodiment of the system, the one or more monitors further include a controller that disconnects the vacuum suction source when the controller detects that the measured physiological indicator of well being is greater or less than predetermined maximum and minimum values for that indicator.

In another embodiment of the system, the vacuum suction source includes a controller which is operative to disconnect the vacuum suction source when the suction produced by the source exceeds a predetermined value for the maximum suction to be applied or when the total duration during which maximum suction has been applied is greater than a predetermined value for the total duration for which the maximum suction is to be applied.

In another embodiment of the system, the one or more second sensors are a plurality of second sensors positioned along the rim of the cup-shaped element.

In yet another embodiment of the system, the one or more second sensors are positioned on a flexible support adjacent to the rim of the cup-shaped element and depressible into the cup-shaped element.

In yet another embodiment of the system, the sensor support has a conductive plate positioned thereon and has a tube integrally formed therewith extending substantially transversally from the plate and the one or more first sensor is configured and sized to be positioned and held within the tube.

In a further embodiment of the system, the system includes a maternal leg plate in electrical communication with the monitor, the maternal signals detected by the leg plate are subtracted, or otherwise eliminated, from the output data obtained from the detected direct and surface wave signals of the fetus.

In another aspect of the present invention, there is provided a method for monitoring the well being of a fetus during a vacuum assisted delivery. The method including the followings steps:
placing a vacuum assisted delivery extractor on the head of a fetus, the extractor having two or more sensors for sensing electrical signals;
activating a vacuum suction source; and
monitoring the well-being of the fetus by detecting and processing signals from both direct waves and surface waves generated by an electrophysiological event in the fetus

In an embodiment of the method, the step of monitoring further includes a step of combining the direct wave and surface wave signals detected by the two or more sensors.

In yet another embodiment of the method, the step of monitoring further includes a step of subtracting, or otherwise eliminating, the maternal signal detected by a maternal leg plate from the output data obtained from the direct and surface wave signals of the fetus.

In still another embodiment of the method, wherein the step of monitoring further includes a step of selecting the cleaner, stronger signal from between the direct and surface wave signals of the fetus after the direct and surface waves signals have had any maternal signal subtracted out, or otherwise eliminated.

In yet another embodiment of the method, the step of activating includes allowing the vacuum suction to reach a first value during the mother's contractions and then allowing the vacuum suction to reach a second value between contractions, where the second value is less than the first value.

In still another embodiment of the method, the method further includes the step of aborting the step of activating a suction source when one or more of the following conditions is indicated: the step of monitoring indicates that the fetus is in distress; the step of monitoring indicates that the maximum suction being applied exceeds a predetermined value for the maximum suction to be applied; and the step of monitoring indicates that the total duration at which the maximum suction has been applied exceeds a predetermined value for the maximum duration.

In an embodiment of the method, the method further includes the step of modulating the strength of the vacuum produced by the vacuum suction source when one or more of the following conditions is indicated: the step of monitoring indicates that the fetus is in distress; the step of monitoring indicates that the maximum suction being applied exceeds a predetermined value for the maximum suction to be applied; the step of monitoring indicates that the total duration at which the maximum suction has been applied exceeds a predetermined value for the maximum duration; and the physician has determined that the vacuum suction strength is to be increased or decreased.

In still another embodiment of the method, the step of placing a vacuum assisted delivery extractor on the head of a fetus, includes positioning against the scalp of the fetus at least two noninvasive sensors for monitoring one or more physiological indicators of the well-being of the fetus, one or more sensors detecting electrical signals of direct waves arising from an electrophysiological event and passing through the fetal skull to the one or more sensors and one or more sensors detecting electrical signals of surface waves arising from the electrophysiological event passing through a wet environment surrounding the fetus, the waves related to the physiological indicators of well being.

In yet another embodiment of the method, the extractor is used as an instrument for internal monitoring of the well being of a fetus when a vacuum-assisted birth is not contemplated and the step of activating activates the suction source at lower pressures than the pressure levels used when a vacuum-assisted birth is contemplated.

In another embodiment of the method, the extractor is used as an external monitoring instrument of the physiological indicators of well being of a patient when the extractor is suctionally attached to the patient's skin. During the step of activating, the vacuum is activated at lower pressure than pressure levels used when a vacuum-assisted birth is contemplated but still sufficient to ensure that the cup-shaped element is attached to the skin of the patient.

In another embodiment of the method, when the skin is dry, the method further includes a step of wetting the skin.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in greater detail than is necessary for a fundamental understanding of the invention. The description taken with the drawings make apparent to those skilled in the art how the several forms of the invention may be embodied in practice.

In the drawings:

FIGS. 1A-1B are schematic isometric and side views of a vacuum assisted delivery extractor in accordance with a first embodiment of the present invention;

FIG. 1C shows a view of the vacuum assisted delivery extractor of FIGS. 1A and 1B with the head of a fetus positioned in the cup of the extractor;

FIGS. 2A-2B are schematic isometric and side views of a vacuum assisted delivery extractor in accordance with a second embodiment of the present invention;

FIGS. 3A-3C are partial schematic isometric and side views of a vacuum assisted delivery extractor in accordance with embodiments of the present invention, the extractor including a means for modulating the strength of the vacuum operative on the fetus;

FIGS. 4A and 4B are side views of yet another embodiment of a vacuum assisted delivery extractor in accordance with yet another embodiment of the present invention;

FIG. 4C is an expanded view of the sensor, sensor housing, sensor support, and wiring of the extractor shown in FIG. 4B;

FIG. 4D shows a top view of the extractor shown in FIGS. 4A-4C;

FIGS. 4E and 4F show cut-away and isometric views of the sensor housing, sensor and the connecting wires of the extractor in FIGS. 4A-4D;

FIGS. 5A and 5B are side views of another embodiment of a vacuum assisted delivery extractor constructed in accordance with the present invention;

FIG. 5C is an expanded view of the sensor, sensor housing, sensor support, and wiring of the extractor shown in FIG. 5B;

FIG. 5D shows a top view of the extractor shown in FIGS. 5A-5C;

FIGS. 5E and 5F show cut-away and isometric views of the sensor housing, sensor and the connecting wires of the extractor in FIGS. 5A-5D;

FIGS. 5G and 5H are side views of yet another scalp electrode embodiment of a vacuum assisted delivery extractor in accordance with the present invention;

FIG. 5I is a side view of the scalp electrode shown in FIG. 5H but having auxiliary scalp attachment elements; and

FIG. 5J is a view of an attachment means for holding the surface wave sensor to the rim of the cup of the extractor.

Similar elements in the Figures are numbered with similar reference numerals.

### DESCRIPTION OF PREFERRED EMBODIMENTS

The present invention provides a vacuum assisted delivery extractor and system equipped with at least one non-invasive sensor. The “sensor” may also be denoted herein as a “monitoring sensor”. Often the sensor is an electrical sensor, also herein referred to at times as a fetal scalp electrode (FSE). The at least one sensor may be used for monitoring inter alia fetal heart rate (FHR) and/or fetal pulse rate (FPR) and/or fetal electrocardiogram (ECG) and/or fetal blood oxygenation (oximetry) during the last stages of delivery, i.e. when the fetus is transiting the birth canal. The above listed physiological indicators for determining the well being of the fetus should be deemed as exemplary only. There is no intent at limiting the physiological indicators which may be measured by a suitable sensor for determining the well-being of the fetus.

It will be appreciated by a person skilled in the art that the sensor used to continuously monitor one physiological indicator need not, and generally will not, be identical to the sensor used to measure/monitor another physiological indicator of fetal well-being. For example, while FHR, FPR, and FECG sensors are typically electrical sensors, an oximetry sensor to continuously monitor fetal oxygen saturation (FSpO₂), that is fetal blood oxygen levels, is typically a photosensor.

While the sensors used herein are described as resilient it should be appreciate that such resilience may flow from the material composition and/or shape of the sensor that is its “inherent” resiliency. On the other hand resiliency may be of an “extrinsic” nature wherein an essentially non-resilient sensor is adapted to be used with a housing having a resilient element, such as a photometric oximeter adapted to have a resilient coil fitted to its end closest to the fetal scalp. Such oximeters are known to persons skilled in the art and commercially available.

The delivery extractor of the present invention is especially useful for monitoring the well being of a fetus during what until now has been a “black out” period, that is, that portion of a delivery when the fetus is transiting the birth canal. The delivery extractor of the present invention is equipped with at least one sensor which provides continuous readings of at least one of the following: FHR, FPR, fetal ECG, fetal blood oxygenation level, and/or other fetal physiological indicators, during this most critical stage of delivery. The reading(s) are intended to indicate possible fetal distress.

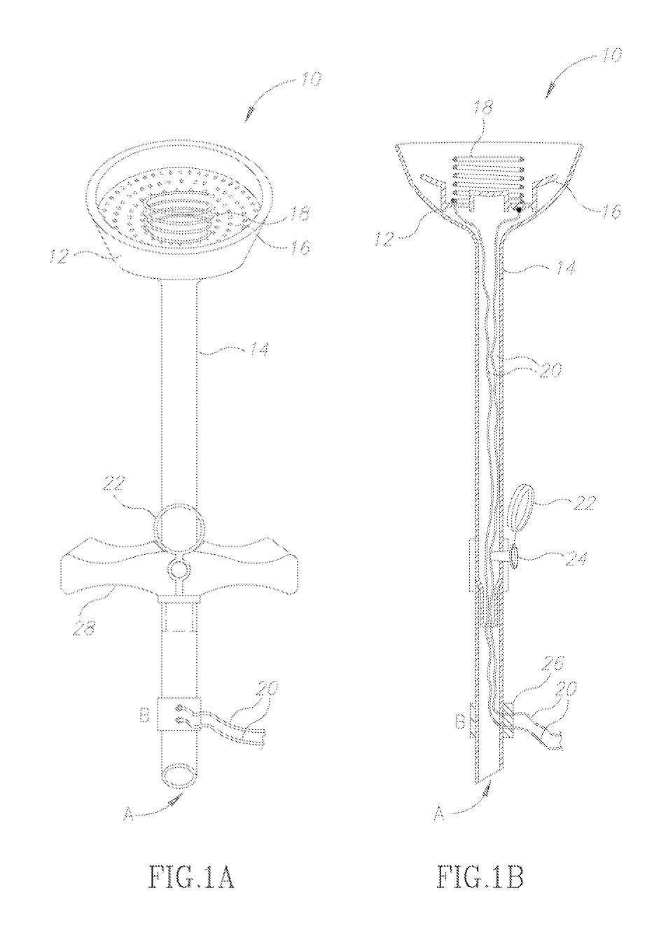

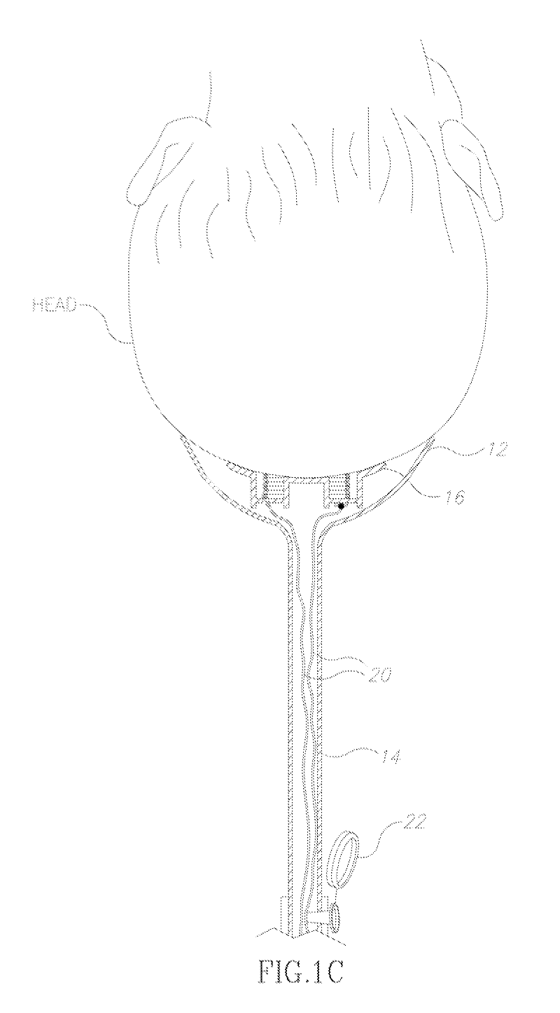

Reference is now made to FIGS. 1A-1C which show a vacuum assisted delivery extractor constructed according to a first embodiment of the present invention. FIGS. 1A and 1B show an isometric and a side view of the vacuum delivery extractor, respectively, while FIG. 1C shows the cup of the extractor positioned on the head of a fetus.

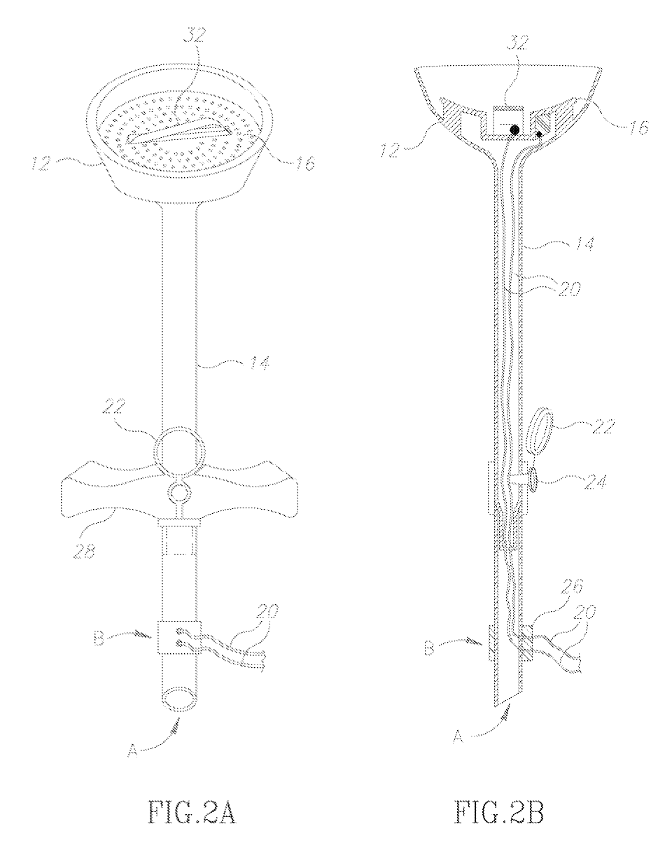

The embodiments shown in FIGS. 1A-2B herein employ electrical sensors suitable for continuously monitoring one or more of the following: FHR, FPR and ECG. With suitable modification of the sensor or with another type of monitoring sensor, the sensor can be used to monitor other physiological indicators of fetal well-being. These include, but should not be construed as being limited to, monitoring blood oxygen levels.

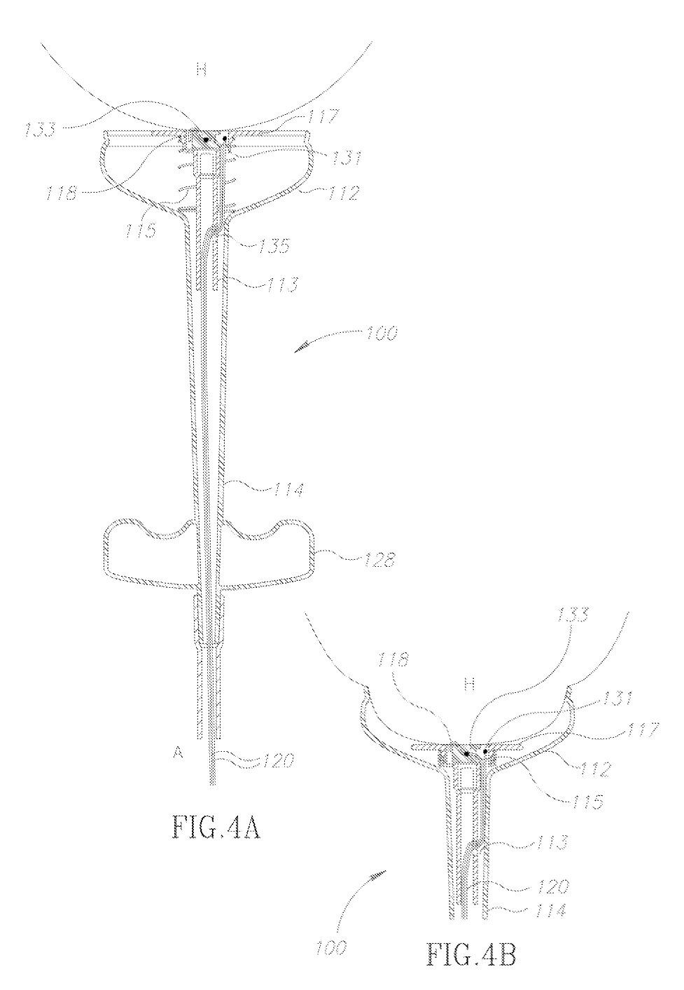

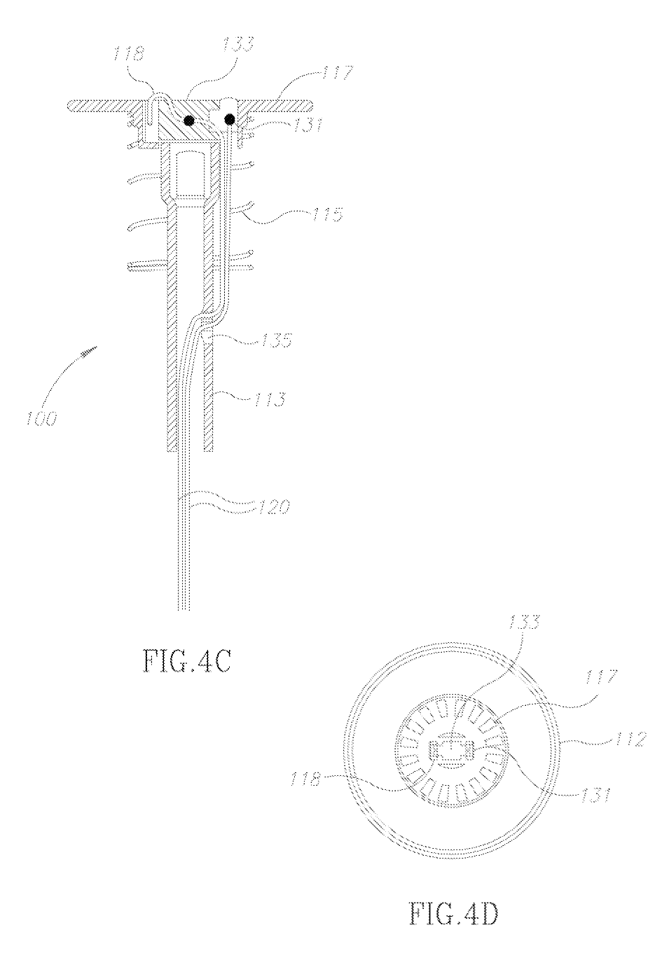

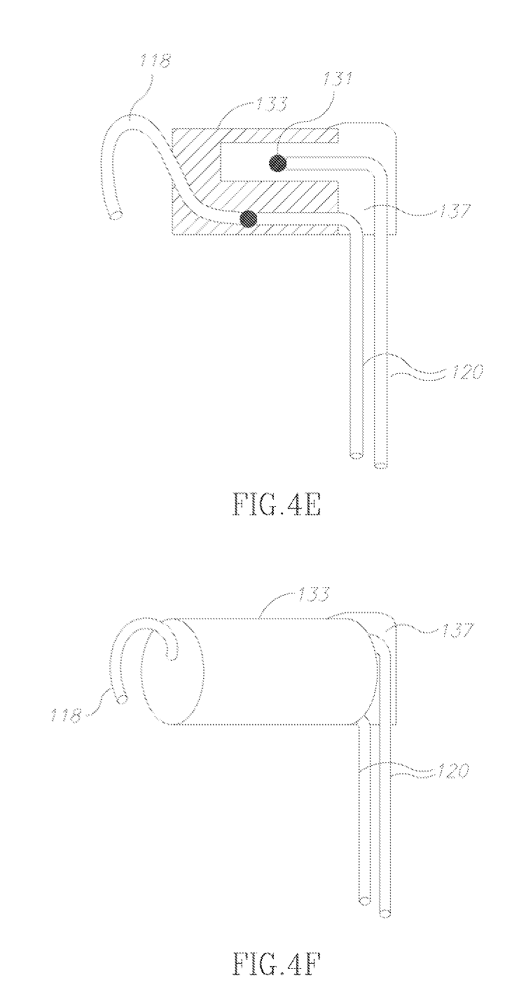

Vacuum delivery extractor 10 includes a cup 12 with a plastic fetal head support 16 positioned therewithin. Fetal head support 16 may also be denoted herein as a sensor support such as element 117 in FIGS. 4A-4E without any intent at differentiating between the construction and operation of the supports except as may otherwise be indicated. A sensor 18 is positioned on and mechanically attached to support 16. Cup 12 is attached to a tubular stem 14, often, but not necessarily, integrally molded to tubular stem 14. Stem 14 may be in mechanical connection with plastic tubing which extends in the direction of end A which is ultimately connected to a vacuum suction source (not shown).

End A of stem 14 may be adapted to be attached to any of many commercially available vacuum suction sources in any of many different ways known to those skilled in the art. Typical commercially available vacuum suction sources that may be used are Dominant 30 or Dominant 50 made by Medella AG, Baar, Switzerland; Senator 30 made by Ardo Medical AG of Unterageri, Switzerland; and S351 Natal made by Atmos Medizin Technik GmBH of Lenzkirch Germany. Typically, vacuum suction sources for use in vacuum assisted deliveries are equipped with gauges and other features to monitor the pressure providing greater safety for the fetus.

Isolated electrically conductive wires attachable to any of many commercially available monitoring devices, such as fetal heart rate (FHR), fetal pulse rate (FPR), and fetal ECG monitoring devices (all of the monitors not shown) are positioned inside the hollow of tubular stem 14. At point B of device 14 there is a hermetic seal 26 which allows wires 20 to exit the hollow of tubular stem 14 without adversely affecting the vacuum suction applied during a delivery.

Isolated electrical conductive wires 20 are brought through the hollow of tubular stem 14 up to and through the concave base of cup 12 and fetal head support 16. One of these two wires is attached to a sensor, typically, but without any intent on limiting other options, an electrical sensor 18 made of a conductive material, typically, but without intending to limit the invention, stainless steel. The second wire is attached to a metal insert (not shown) in support 16 and serves as a grounding potential for the circuit, neutralizing background noise. Sensor 18 is permanently affixed within cup 12 on the side of cup 12 distal from tubular stem 14. Sensor 18 is typically constructed to be flexible and resilient.

Sensor 18 may have resilient helical (spiral), double helical and clip-like shapes. It should readily be understood by those skilled in the art that the sensors may have shapes other than those listed above. The shapes listed above are to be considered exemplary only and not limiting.

The nature of the measurement being made may determine the nature and shape of the sensor used. For example, an oximetry sensor may be substantially different from a sensor used in FPR, FHR, or ECG monitoring. For FPR and FHR monitoring, the construction of the sensor, not necessarily its sheath, may be very similar to that used with today's FSEs. For an oximetry sensor, the sensor may be a photosensor.

As noted, but as examples only and without any intent at limiting the invention, these measurements include one or more of the following: FHR, FPR, ECG, and oximetry. If more than a single measurement is being made concurrently, more than one sensor 18 or more than one type of sensor may be present and more than a single pair of wires 20 may be used.

Extractor 10 allows medical personnel attending the delivery to read/view or hear the heart beat or pulse or ECG or blood oxygen level of the fetus on the at least one external monitor (not shown) in electrical or other communication with extractor 10.

Extractor 10 includes a finger grip 28 which is attached to stem 14. Finger grip 28 is used to position extractor 10, as required, during delivery.

A typical, but non-limiting, monitor that may be used is the Philips Hewlett-Packard M1350A Fetal Monitor available from Somas Technology Inc., Cheshire, Conn. Typical, but non-limiting, connectors, adaptors, cables and transducers for use in joining the above monitor to wires 20 extending from sensor 18 are similar to those supplied by Phillips for use with the above monitor M1350A and their fetal scalp electrodes, for example, electrode 15133E.

The electronics required to integrate the sensors and monitors is well known to persons skilled in the art. Such integration, for example, is regularly found in systems employing currently available FSEs with their associated monitors or ECG sensors with their associated monitors. Accordingly, the electronics interface necessary between the at least one monitor used with a vacuum assisted delivery extractor constructed according to the present invention and its associated sensor(s) can readily be developed by persons skilled in the art. Only minor and inconsequential modifications may be required.

Similarly, the sensors for use in FHR and FPR monitoring according to the present invention are very similar in construction to currently commercially available FSEs. Little or no modification would be required of their electronics. Sensors which can be used include Philips Medizin Systeme (Boblingen, Germany) FSEs Models 15133D and 15133E and Utah Medical Products Inc (Midvale, Utah) Qwik Connect Plus™ FSE (product number FSE-500P).

Extractor 10 has an emergency suction reduction means 22, here shown as a pull tab, which is formed to be in association with an emergency plug 24 that fits into an aperture in stem 14. When during delivery the fetus is not experiencing difficulty, emergency suction reduction means 22, here a pull tab, associated with plug 24, allows plug 24 to remain in its aperture in stem 14 forming a vacuum seal therewith, allowing suction to be maintained. If sensor 18 indicates that the fetus is experiencing difficulty during the delivery, the doctor, midwife or other health professional may pull on emergency suction reduction means 22 causing plug 24, typically integrally formed with tab 22, to fall out of its aperture breaking the vacuum. The delivery can then be aborted and a caesarean section performed; alternatively, other medical procedures may be applied to the fetus experiencing difficulty.

Sensor 18 is positioned on fetal head support 16. Both cup 12 and fetal head support 16 are formed to have substantially concave profiles with the latter nested in the former. The concavity allows the fetal crown to fit into cup 12. When a vacuum suction source (not shown) is attached to end A of stem 14 and activated, the fetal head further compresses resilient sensor 18 against fetal head support 16 and the bottom of cup 12 ensuring good contact. This allows measurements, typically, but without being limiting, electrical measurements, to be made and signals to be sent to the at least one monitor. Inter alia, ECG, pulse and/or heart rate readings are derivable from the measurements obtained by sensor 18 and the signals they generate.

Sensor 18 is positioned on fetal head support 16 so that it lies substantially at the center of support 18 and substantially at the center of cup 12. As seen from FIGS. 1A-2B the sensor may also be thought to lie generally on an axis extending from tubular stem 14 through the center of support 18.

Sensor 18 is attached, typically flexibly attached, to fetal head support 16, which is typically formed from a flexible resilient plastic material. Additionally, because of the resilient material from which head support 16 is formed, it is itself compressible within cup 12. Support 18 moves further into the cup when the fetal head presses on it after activation of the vacuum source.

While what has been described above discusses the conductive isolated wires as being conducted to the outside of stem 14 of extractor 10 through hermetic seal 26 to one or more monitors (not shown), wires 20 can conduct signals to a wireless transmitter which can wirelessly communicate with a wireless receiver in electrical communication with one or more monitors. The wireless transmitter (not shown) may be positioned at various sites in extractor 10, for example, inside the hollow of tubular stem 14 or inside finger grip 28.

Reference to FIGS. 2A and 2B is now made. The embodiment here is very much like the embodiment discussed above in FIGS. 1A-1C. The difference is that resilient sensor 18 instead of being shaped as a coil is shaped as a bent leaf spring or resilient clip 32.

Sensor 18 of the present invention is able to provide signals immediately upon being compressed by the head of the fetus held by suction in cup 12. Thus even when the vacuum is less than the vacuum required to accelerate delivery, the latter vacuum typically being on the order of about 600 mm Hg, sensor readings may be obtained.

It is envisioned that embodiments of the present invention will allow for a warning to be emitted if sensor 18 provides a reading/signal above or below a predetermined value. The warning will indicate to the doctor, midwife or other health professional, that the fetus may be in distress or that a possible dangerous operating condition has developed. The warning can be, for example, an audible warning, a visible warning or any other type of warning. The warning element of the system may be positioned within the monitor attached to extractor 10 or in a separate unit of the system.

In the embodiments discussed in conjunction with FIGS. 1A-2B only a single sensor is shown and discussed. In other embodiments of the present invention there may be more than a single sensor positioned in cup 12. The plurality of sensors may be of the same type measuring the same physiological indicator or they may be of different types measuring different types of physiological indicators. When a plurality of sensors is used and multiple measurements or multiple types of measurements are made, they may be made concurrently or separately and in series.

In the embodiments discussed in conjunction with FIGS. 1A-2B only a single pair of wires is discussed. The present invention also contemplates the possibility that multiple pairs of wires may be connected to a sensor or multiple sensors providing input to multiple monitors, including multiple different types of monitors.

In the embodiments discussed above the sensor has been discussed as spiral, coiled, or leaf-like shaped. It will be appreciated, however, that the shape of the sensor may be any shape that allows for good electrical contact with the scalp of the fetus.

In the embodiments discussed above, extractor 10 is formed of plastic materials except for sensor 18 or 32, wires 20, and the metal insert discussed immediately below. When sensor 18 or 32 is an electrical sensor, it and the wire 20 connected to it and the wire 20 connected to a metal insert (not shown) in support 16 are formed of electrically conductive material. Typical plastics which may be used for forming the remainder of the extractor, and individual pieces thereof when the extractor is not integrally molded are high density polyethylene (HDPE), polypropylene, acrylonitrile-butadiene-styrene (ABS) and the like.

When multiple plastic pieces are required, such as when joining the cup to the tubular stem 14 or the tubular stem 14 to the vacuum suction source, the joins are hermetic to ensure production of sufficient suction to assist in delivering the fetus.

Oximetry sensors are known in the art and they may be used in embodiments of the present invention. At times, slight modification may be required but this can readily be effected by persons skilled in the art. One such oximetry sensor and sensor electronics which can be adapted by a person skilled in the art for use with the present invention can be found in U.S. Pat. No. 4,773,422 to Isaacson et al, the contents of which are incorporated herein by reference.

It is to be remembered that sensor 18 or 32 of the present invention is non-invasive and, when used, need only contact the scalp of the fetus. This prevents infections of the scalp as often occurs with other electrodes presently known.

The present invention is envisioned as allowing easy placement for occiput posterior or occiput transverse births.

The present invention is typically a single-use, disposable device.

The cup may be formed having a standard diameter size, such as having a 6 inch diameter, or alternatively, the extractor may be constructed to allow for use with cups of different sizes. The size of the cup discussed here is exemplary only and is not intended to limit the invention.

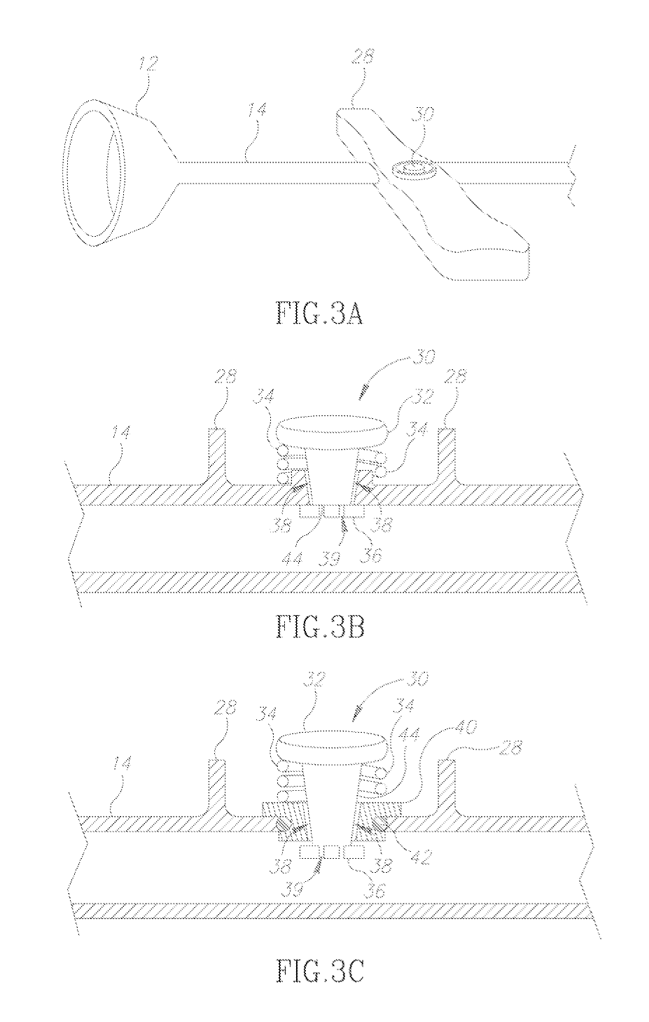

Reference is now made to FIGS. 3A-3C where two additional embodiments of the present invention are shown. Instead of the emergency suction reduction means 22 in FIGS. 1A-2B wherein the emergency suction reduction means is a pull tab emergency plug, the embodiments in FIGS. 3A-3C show a turbo button positioned in the tubular stem 14 at the level of finger grip 28. The turbo button 30 functions as a vacuum suction modulating means modulating the strength of the vacuum at the discretion of the user, typically a physician. In FIGS. 3A-3C, the one or more sensors 18 in cup 12 and the wires 20 within stem 14 are not shown as the Figure is intended to emphasize the positioning of turbo button 30. The remainder of the extractor elements in these Figures are substantially as shown in and described in conjunction with FIGS. 1A-2B.

FIG. 3B is a partial side view of a vacuum delivery extractor constructed according to the present invention including a turbo button 30. The elements shown in the Figure are all formed from plastic except for a spring element 34 typically constructed from stainless steel. When button 30 is not pressed by a finger of the user positioned in the extractor's finger grip 28, button 30 allows a small quantity of air to enter tubular stem 14 via air passages 38 formed by the conical portion 44 of button 30 and the walls of stem 14, reducing the strength of the vacuum, typically, by only 25-50 mm Hg. Air passing through passages 38 continues through slots 39 in retaining button 36 of turbo button 30 into stem 14. When the user wishes to increase the vacuum in the tube stem 14, he presses cap 32 of turbo button 30 so that button 30 moves into the aperture (analogous to referenced element 24 in FIGS. 1B and 2B) in stem 14, closing off passages 38 and increasing the vacuum within stem 14, typically, to a maximum of about 600 mm Hg.

FIG. 3C is a partial side view of yet another embodiment of a vacuum delivery extractor constructed according to the present invention and having a turbo plug. The elements shown in the Figure are of plastic except for a spring element 34, typically but without intending to limit the invention, constructed from stainless steel and a sleeve 40 also typically formed from stainless steel. Sleeve 40 is sealed tight against tubular stem 14 with an O-ring 42 typically formed of an elastomeric material. When turbo button 30 is not pressed by a finger positioned in the extractor's finger grip 28, button 30 allows a small quantity of air to enter tubular stem 14 via air passages 38 formed by the conical portion 44 of turbo button 30 and the walls of stem 14 reducing the strength of the vacuum, typically by only 25-50 mm Hg. Air passing through passages 38 continues through slots 39 in retaining button 36 of turbo button 30 into the inside of tubular stem 14. When the user wishes to increase the vacuum in tubular stem 14 of the extractor, the user presses cap 32 of turbo button 30 so that button 30 moves into the aperture in stem 14 and passages 38 are sealed off by the walls of the conical portion 44 of button 30 and the vacuum within stem 14 increases, typically, to a maximum of about 600 mm Hg.

In embodiments of both FIGS. 3B and 3C, when the turbo button is in its normal unpressed open position the vacuum is typically, but without intending to limit the invention, 550-575 mm Hg and when the turbo button is pressed the pressure rises typically to 600 mm Hg.

Reference is now made to FIGS. 4A-4F where yet another embodiment of a vacuum assisted delivery extractor constructed according to the present invention is shown. FIGS. 4A and 4B each show side views of the extractor. In FIG. 4A, the fetus's head H is brought proximate to the extractor's sensor support while in FIG. 4B the fetus's head H contacts the extractor's sensor and pushes the sensor support into the extractor cup. FIG. 4C is an expanded view of the sensor, sensor housing, sensor support and connecting wires thereto shown in FIG. 4B while FIG. 4D is a top view of the extractor in FIGS. 4A-4C. FIGS. 4E and 4F are two views of the sensor, sensor housing, and connecting wires thereto.

Vacuum assisted delivery extractor 100 includes a plastic cup 112 having a sensor support 117 positionable within it. The sensor support 117 serves both as the fetal head support discussed in previous embodiments and as a support for a sensor as discussed below. In previous embodiments, the support is typically formed of a flexible plastic whereas in the present embodiment it is made of a rigid plastic. In this embodiment as in previous embodiments, the fetus's head H contacts sensor 118 and pushes against sensor support 117 so that support 117 moves further into cup 112 as in FIG. 4B.

Cup 112 is attached to a tubular stem 114, often, but not necessarily, integrally molded to it. Cup 112 and sensor support 117 are typically formed of rigid or semi rigid plastics, such as, for example, high density polyethylene (HDPE), polypropylene, and acrylonitrile-butadiene-styrene (ABS).

Stem 114 may be in mechanical connection with plastic tubing which extends in the direction of end A. End A is ultimately coupled to a vacuum suction source (not shown). End A of tubular stem 114 may be adapted to be attached to any of many commercially available vacuum suction sources in any of many different ways known to those skilled in the art. Typical commercially available vacuum suction sources that may be used have been discussed herein above in conjunction with previous embodiments.

A pair of isolated electrically conductive wires 120 attachable to any of many commercially available monitoring devices, such as fetal heart rate (FHR), fetal pulse rate (FPR), and fetal ECG monitoring devices (not shown) are positioned inside the hollow of tubular stem 114. At a point (not shown) of stem 114 there may be a hermetic seal (not shown) which allows wires 120 to exit the hollow of tubular stem 114 without adversely affecting the vacuum suction applied during a delivery.

Isolated electrical conductive wires 120 are brought through the hollow of tubular stem 114 up to and through plastic tube 113. Tube 113, extending substantially transversally from support 117, may be in mechanical connection or integrally formed with sensor support 117. Wires 120 typically exit through an opening 135 in a wall of tube 113 to be threaded between tube 113 and tubular stem 114 bringing them to sensor housing 133. One of the wires of the pair of wires 120 serves as a grounding potential for neutralizing background noise, and is soldered 131A to a conductive plate 137 best seen in FIG. 4E. The algorithm for obtaining a measurement of the desired physical parameter is operative when both sensor 118 and conductive plate 137 contact the scalp of the fetus. Conductive plate 137 is formed of an electrically conductive material, such as stainless steel, and positioned in sensor housing 133 by any of many procedures known to those skilled in the art, for example by insert molding or by applying pressure. Sensor housing 133 is typically formed of a rigid plastic.

The other wire of the pair of wires 120 passes through sensor housing 133 and is in electrical contact with an electrically conductive sensor 118 (best seen in FIGS. 4C and 4E) made of a conductive material, typically, stainless steel. Sensor 118 may be permanently affixed on one side of sensor housing 133 and soldered at point 131B to one of wires 120.

Sensor housing 133 is typically recessed within sensor support 117 forming a substantially flat surface therewith as best seen in FIG. 4C.

External to tube 113 and substantially coaxial with it is a spring 115 whose purpose is solely to provide mechanical support to and proper positioning of rigid tube 113 so that it sits substantially collinear with tubular stem 114. The extended and compressed states of spring 115 are shown in FIGS. 4A and 4B respectively.

Sensor 118 may be constructed to be flexible and resilient. Sensor 118 may have resilient helical (spiral), double helical, coiled and clip shapes. It should readily be understood by those skilled in the art that sensor 118 may have shapes other than those listed above. The shapes listed above are to be considered exemplary only and not limiting. In general, the shape of the sensor may be any shape that allows for good electrical contact with the scalp of the fetus without penetrating the scalp.

Sensor 118 is positioned in sensor housing 133 which is situated in the sensor support 117. Cup 112 is typically formed to have a substantially concave profile while sensor support 117 is formed to have substantially flat profile. The concavity of cup 112 allows for the fetal crown to at least partially fit into cup 112. Sensor support 117 is translationally movable within cup 112 in the general direction of the end A of tubular stem 114. As indicated in the Figures, sensor 118 is positioned so that it is located generally in the center of support 117 and cup 112 substantially along an axis running through tube 113 and the center of support 117. This is similar to the positioning of sensor 18 in previous embodiments.

When a vacuum suction source (not shown) is attached to end A of tubular stem 114 and activated, the fetal head contacts sensor support 117 and compresses mechanical spring 115. This also pushes sensor support 117 further into cup 112 ensuring good contact between sensor 118 and the fetal scalp. This allows electrical measurements to be made and signals to be sent to the at least one monitor.

Mechanical spring 115 shown in FIGS. 4A-4C allows sensor support 117 to be compressed in the direction of tubular stem 114 as shown in FIG. 4B. This is functionally similar to fetal head support 16 (FIGS. 1A-2B) which because of its inherent flexibility and resiliency is compressible within cup 12 (FIGS. 1A-2B) when the head of the fetus is drawn into cup 12 by the vacuum.

As in previous embodiments, extractor 100 includes a finger grip 128 which is attached to tubular stem 114. Finger grip 128 is used to position extractor 100, as required, during delivery.

Extractor 100 allows medical personnel attending the delivery to monitor the heart beat or pulse or ECG of the fetus on the at least one external monitor (not shown) in electrical or other communication with extractor 100. A typical, but non-limiting, monitor as well as typical, but non-limiting, connectors, adaptors, cables and transducers for use in joining the above monitor to wires 120 extending from sensor 118 have been described herein above in conjunction with previous embodiments.

The electronics required to integrate the sensors and monitors is well known to persons skilled in the art. Such integration, for example, is regularly found in systems employing currently available FSEs with their associated monitors or ECG sensors with their associated monitors. Accordingly, the electronics interface necessary between the at least one monitor used with a vacuum assisted delivery extractor 100 and its associated sensor(s) can readily be developed by persons skilled in the art. Only minor and inconsequential modifications may be required.

Similarly, the sensors for use in FHR and FPR monitoring according to the present invention may be similar in construction to currently commercially available FSE's. Little or no modification would be required of their electronics. Typical sensors have been described herein above in conjunction with previous embodiments.

As in previous embodiments, extractor 100 may be equipped with an emergency suction reduction means (not shown) which can be constructed and operated as described herein above.

While what has been described above discusses the conductive isolated wires as being conducted to the outside of stem 114 of extractor 100 through a hermetic seal (not shown) to one or more monitors (also not shown), wires 120 can conduct signals to a wireless transmitter which can wirelessly communicate with a wireless receiver in electrical communication with one or more monitors. The wireless transmitter (not shown) may be positioned at various sites in extractor 100, for example, inside the hollow of tubular stem 114 or inside finger grip 128.

As in previous embodiments, it is envisioned that the embodiment shown in FIGS. 4A-4F will allow for a warning to be emitted if sensor 118 provides a reading/signal above or below a predetermined value. The warning will indicate to the doctor, midwife or other health professional, that the fetus may be in distress or that a possible dangerous operating condition has developed.

In the embodiment discussed in conjunction with FIGS. 4A-4F only a single sensor 118 is shown and discussed. In other embodiments of the present invention there may be more than a single sensor positioned in cup 112 and more than a single measurement concurrently. The plurality of sensors may be of the same type measuring the same physiological indicator or they may be of different types measuring different types of physiological indicators. When a plurality of sensors is used and multiple measurements or multiple types of measurements are made, they may be made concurrently or separately and in series.

In the embodiment discussed in conjunction with FIGS. 4A-4F only a single pair of wires is discussed. The present invention also contemplates the possibility that multiple pairs of wires may be connected to a sensor or multiple sensors providing input to multiple monitors, including multiple different types of monitors.

In the embodiment illustrated in FIGS. 4A-4F discussed above, extractor 100 is typically formed of plastic materials except for sensor 118 and wires 120. Sensor 118 being an electrical sensor, and wires 120 connected to it are formed of electrically conductive material. Typical plastics which may be used for forming the remainder of the extractor, and individual pieces thereof when the extractor is not integrally molded, are high density polyethylene (HDPE), polypropylene, acrylonitrile-butadiene-styrene (ABS) and the like.

When multiple plastic pieces are required, such as when joining the cup to the tubular stem 114 or the tubular stem 114 to the vacuum suction source, the joins are hermetic to ensure production of sufficient suction to assist in delivering the fetus.

Sensor 118 of the present embodiment is an active non-invasive sensor, which when used, need only contact the scalp of the fetus. This prevents infections of the scalp as often occurs with other electrodes presently known.

As with previous embodiments, the present embodiments are envisioned as allowing easy placement for occiput posterior or occiput transverse births.

As in previous embodiments, the embodiment shown in FIGS. 4A-4F is also envisioned as a single-use device. Similarly, as in previous embodiments, the extractor may be constructed to allow for use with a standard diameter cup or with cups of different sizes.

It is anticipated that the present invention will be widely used by physicians and hospitals as a means of practicing “defensive” medicine thereby contributing to a reduction in malpractice insurance.

The present invention also provides a method for monitoring the well being of a fetus during a vacuum assisted delivery. The method includes the followings steps.
placing a vacuum assisted delivery extractor on the head of a fetus as it enters the birth canal, thereby positioning against the scalp of the fetus at least one non-invasive monitoring sensor for monitoring one or more physiological indicators of the well-being of the fetus;
activating a vacuum suction source during the mother's contractions for assisting movement of the fetus along the birth canal; and
monitoring the one or more physiological indicators of the well-being of the fetus via the at least one non-invasive monitoring sensor as the fetus moves along the birth canal.

The method may also include the step of discontinuing the vacuum suction source when the mandible of the fetus passes the mother's pubic symphysis.

The step of placing typically occurs after a physician has digitally determined that the fetus is entering the birth canal.

The step of activating includes allowing the suction to reach a first value during the mother's contractions and then allowing the suction to reach a second value between the mother's contractions where the second value is less than the first value. Typically, the first value is about 600 mm Hg while the second value is typically, but without being limiting, about 400 mm Hg.

In other embodiments which reflect other hospital protocols, the step of activating requires that the vacuum suction be progressively increased until a maximum predetermined value is reached. This value is maintained until the head of the fetus exits the birth canal and or the vacuum assisted delivery is otherwise aborted by the physician.

The method may also include a further step, a step of aborting. The step of aborting aborts the step of activating a suction source when one or more of the following conditions is indicated: the step of monitoring indicates that the fetus is in distress; the step of monitoring indicates that the maximum suction exceeds a predetermined value for the maximum suction which is to be applied; and the step of monitoring indicates that the total duration during which the maximum suction has been applied exceeds a predetermined value for the maximum duration.

The present invention also teaches another method for monitoring the well-being of a fetus during a vacuum assisted delivery. The method comprising the steps of:
placing a vacuum assisted delivery extractor on the head of a fetus as it enters the birth canal, thereby to position against the scalp of the fetus at least one non-invasive monitoring sensor for monitoring one or more physiological indicators of the well-being of the fetus;
activating a vacuum suction source during the mother's contractions for assisting movement of the fetus along the birth canal and to hold the monitoring sensor in sensory contact with the scalp of the fetus; and
monitoring the one or more physiological indicators of the well-being of the fetus via the at least one non-invasive monitoring sensor as the fetus moves along the birth canal.

The “sensory contact” noted in the step of activating above and in the “contact” discussed in the claims does not always require physical contact. In some cases physical proximity will suffice. It does however require that the distance of the sensor from the scalp allow for sensing and monitoring of the desired physiological indicator of fetal well-being. In cases where the sensor is an electrical sensor, actual physical contact may typically be required. In other cases, such as with oximetry sensors, no physical contact with the scalp may be required and proximity of the sensor to the scalp alone will suffice.

This second method of the present invention includes an embodiment wherein the step of activating occurs during the mother's contractions and assists in moving the fetus along the birth canal. It also includes an embodiment wherein the step of activating includes allowing the suction to reach a first value during the mother's contractions and then allowing the suction to reach a second value between the mother's contractions where the second value is less than the first value. Typically, the first value is about 600 mm Hg while the second value is typically, but without limiting the invention, about 400 mm Hg.

In other embodiments which reflect other hospital protocols the step of activating requires that the vacuum suction be progressively increased until a maximum predetermined value is reached. This value is maintained until the head of the fetus exits the birth canal and or the vacuum assisted delivery is otherwise aborted by the physician.

In an embodiment of the second method, the method further includes the step of aborting. The step of aborting aborts the step of activating a suction source when one or more of the following conditions is indicated: the step of monitoring indicates that the fetus is in distress; the step of monitoring indicates that the maximum suction exceeds a predetermined value for the maximum suction to be applied; or the step of monitoring indicates that the total duration at which the maximum suction has been applied exceeds a predetermined value for the maximum duration.

In another embodiment of the second method, the method further includes the step of modulating the strength of the vacuum produced by the vacuum suction source when at least one of the following conditions is indicated: the step of monitoring indicates that the fetus is in distress; the step of monitoring indicates that the maximum suction being applied exceeds a predetermined value for the maximum suction to be applied; the step of monitoring indicates that the total duration at which the maximum suction has been applied exceeds a predetermined value for the maximum duration; and/or the physician has determined that the vacuum suction strength is to be increased or decreased.

It is expected that the extractor, extractor system and method of the present invention will reduce the risk of the well-known “cone head” and hematoma phenomena associated with vacuum assisted deliveries. Similarly, it is expected that the extractor, extractor system and method of the present invention will reduce incidences of brain damage and/or more quickly detect fetal distress.

It should be appreciated that in the description herein and in the claims below, whenever the user is described by the term “physician”, the term “physician” is meant to also apply to a midwife or other health care professional.

The present invention also provides a method for monitoring the well being of a fetus during a vacuum assisted delivery. The method includes the followings steps.
placing a vacuum assisted delivery extractor on the head of a fetus as it enters the birth canal, thereby positioning against the scalp of the fetus at least one non-invasive monitoring sensor for monitoring one or more physiological indicators of the well-being of the fetus;
activating a vacuum suction source during the mother's contractions for assisting movement of the fetus along the birth canal; and
monitoring the one or more physiological indicators of the well-being of the fetus via the at least one non-invasive monitoring sensor as the fetus moves along the birth canal.

The method may also include the step of discontinuing the vacuum suction source when the mandible of the fetus passes the mother's pubic symphysis.

The step of placing typically occurs after a physician has digitally determined that the fetus is entering the birth canal.

The step of activating includes allowing the suction to reach a first value during the mother's contractions and then allowing the suction to reach a second value between the mother's contractions where the second value is less than the first value. Typically, the first value is about 600 mm Hg while the second value is typically, but without being limiting, about 400 mm Hg.

In other embodiments which reflect other hospital protocols, the step of activating requires that the vacuum suction be progressively increased until a maximum predetermined value is reached. This value is maintained until the head of the fetus exits the birth canal and or the vacuum assisted delivery is otherwise aborted by the physician.

The method may also include a further step, a step of aborting. The step of aborting aborts the step of activating a suction source when one or more of the following conditions is indicated: the step of monitoring indicates that the fetus is in distress; the step of monitoring indicates that the maximum suction exceeds a predetermined value for the maximum suction which is to be applied; and the step of monitoring indicates that the total duration during which the maximum suction has been applied exceeds a predetermined value for the maximum duration.

The present invention also teaches another method for monitoring the well-being of a fetus during a vacuum assisted delivery. The method comprising the steps of:
placing a vacuum assisted delivery extractor on the head of a fetus as it enters the birth canal, thereby to position against the scalp of the fetus at least one non-invasive monitoring sensor for monitoring one or more physiological indicators of the well-being of the fetus;
activating a vacuum suction source during the mother's contractions for assisting movement of the fetus along the birth canal and to hold the monitoring sensor in sensory contact with the scalp of the fetus; and
monitoring the one or more physiological indicators of the well-being of the fetus via the at least one non-invasive monitoring sensor as the fetus moves along the birth canal.

The “sensory contact” noted in the step of activating above and in the “contact” discussed in the claims does not always require physical contact. In some cases physical proximity will suffice. It does however require that the distance of the sensor from the scalp allow for sensing and monitoring of the desired physiological indicator of fetal well-being. In cases where the sensor is an electrical sensor, actual physical contact may typically be required. In other cases, such as with oximetry sensors, no physical contact with the scalp may be required and proximity of the sensor to the scalp alone will suffice.

This second method of the present invention includes an embodiment wherein the step of activating occurs during the mother's contractions and assists in moving the fetus along the birth canal. It also includes an embodiment wherein the step of activating includes allowing the suction to reach a first value during the mother's contractions and then allowing the suction to reach a second value between the mother's contractions where the second value is less than the first value. Typically, the first value is about 600 mm Hg while the second value is typically, but without limiting the invention, about 400 mm Hg.

In other embodiments which reflect other hospital protocols the step of activating requires that the vacuum suction be progressively increased until a maximum predetermined value is reached. This value is maintained until the head of the fetus exits the birth canal and or the vacuum assisted delivery is otherwise aborted by the physician.

In an embodiment of the second method, the method further includes the step of aborting. The step of aborting aborts the step of activating a suction source when one or more of the following conditions is indicated: the step of monitoring indicates that the fetus is in distress; the step of monitoring indicates that the maximum suction exceeds a predetermined value for the maximum suction to be applied; or the step of monitoring indicates that the total duration at which the maximum suction has been applied exceeds a predetermined value for the maximum duration.

In another embodiment of the second method, the method further includes the step of modulating the strength of the vacuum produced by the vacuum suction source when at least one of the following conditions is indicated: the step of monitoring indicates that the fetus is in distress; the step of monitoring indicates that the maximum suction being applied exceeds a predetermined value for the maximum suction to be applied; the step of monitoring indicates that the total duration at which the maximum suction has been applied exceeds a predetermined value for the maximum duration; and/or the physician has determined that the vacuum suction strength is to be increased or decreased.

It is expected that the extractor, extractor system and method of the present invention will reduce the risk of the well-known “cone head” and hematoma phenomena associated with vacuum assisted deliveries. Similarly, it is expected that the extractor, extractor system and method of the present invention will reduce incidences of brain damage and/or more quickly detect fetal distress.

It should be appreciated that in the description herein and in the claims below, whenever the user is described by the term “physician”, the term “physician” is meant to also apply to a midwife or other health care professional.

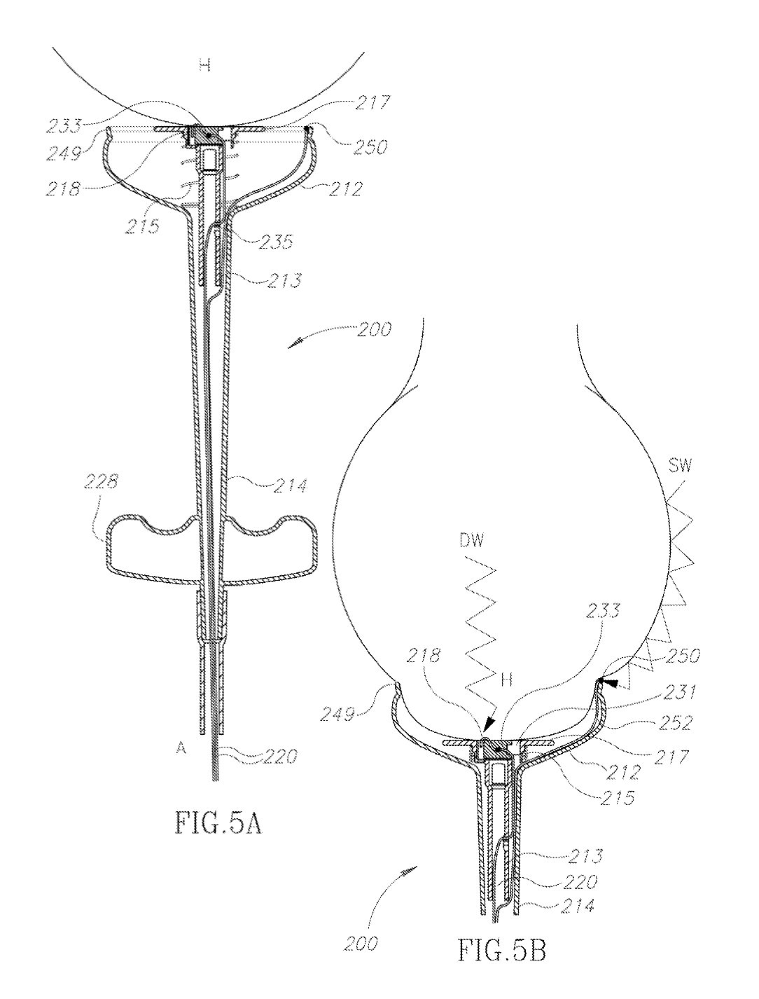

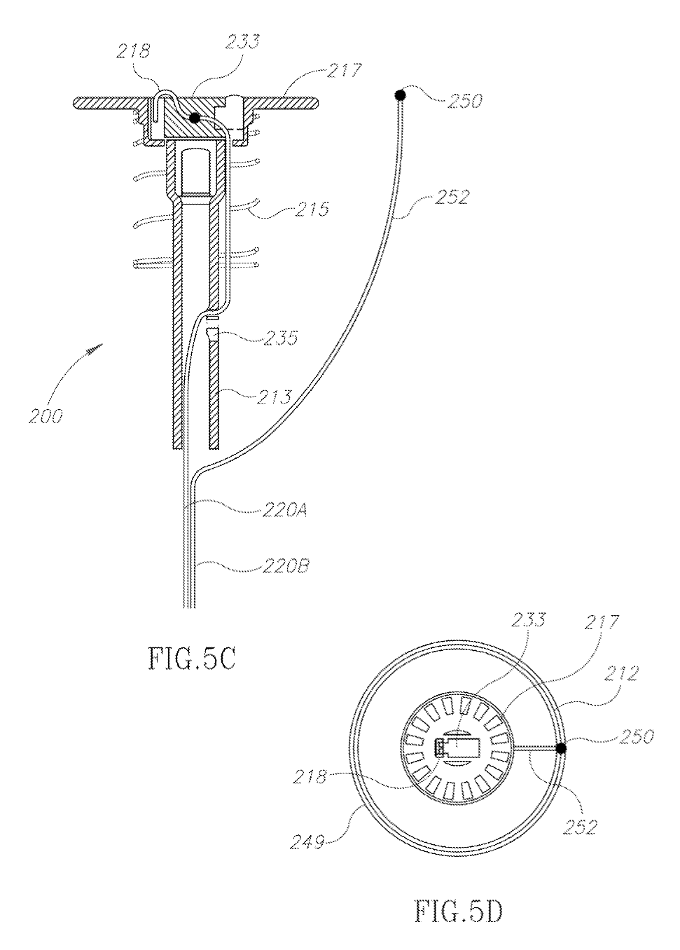

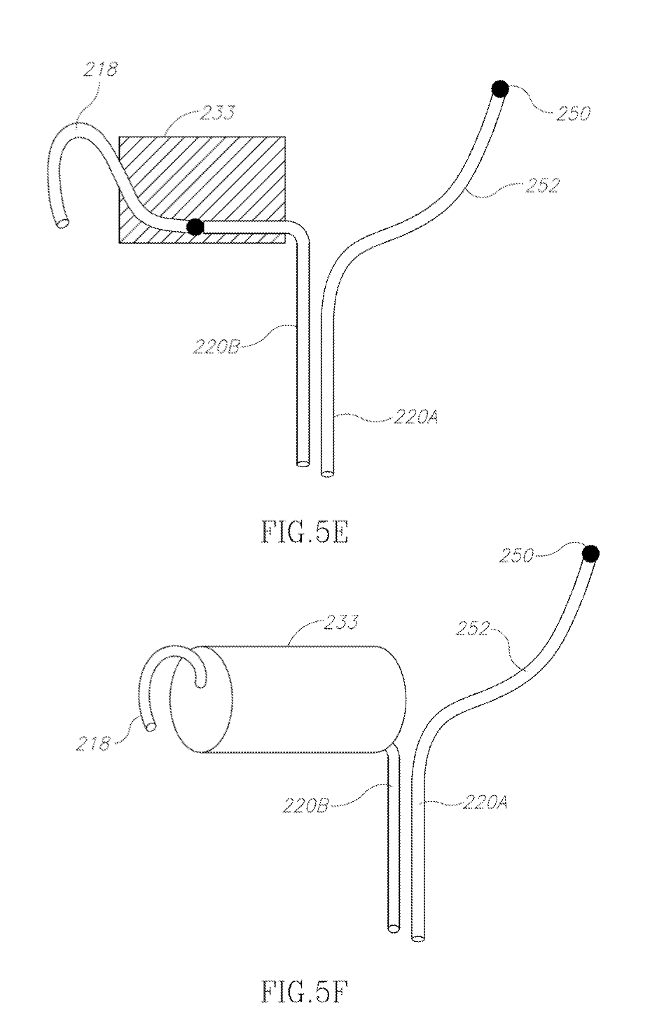

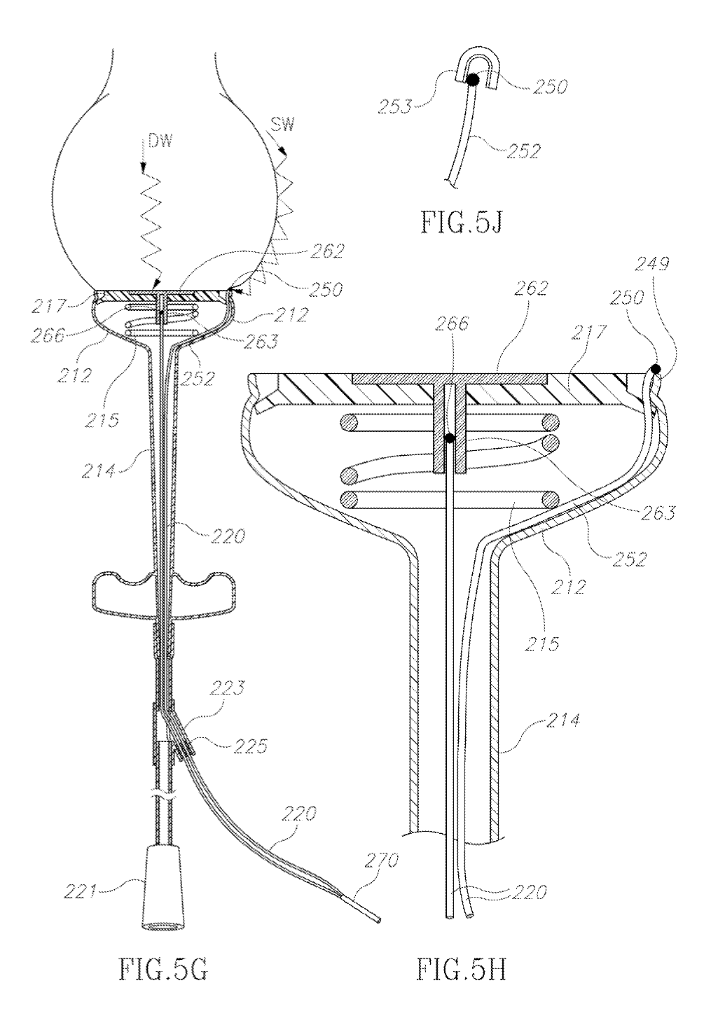

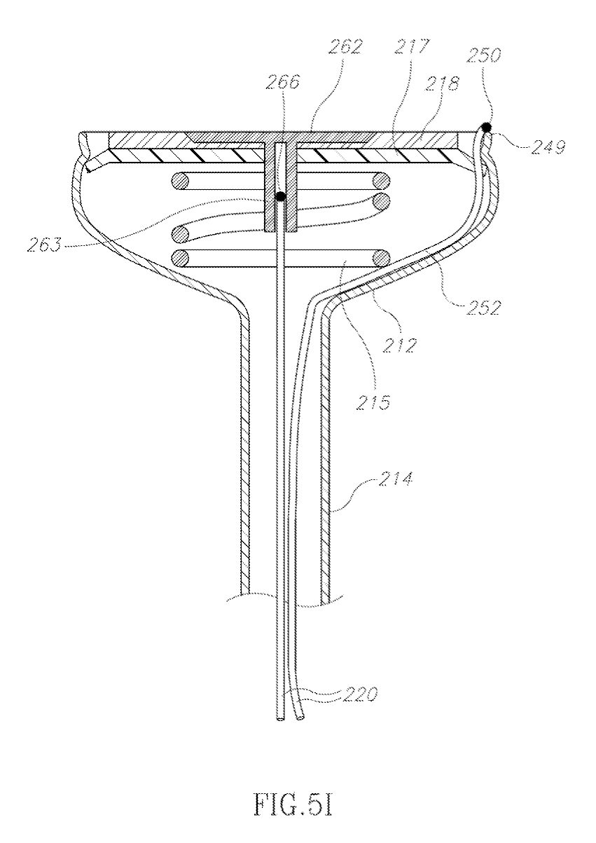

Reference is now made to FIGS. 5A-5F where yet another embodiment of a vacuum assisted delivery extractor constructed according to the present invention is shown. FIGS. 5A and 5B each show side views of the extractor. In FIG. 5A, the fetus's head H is brought proximate to the extractor's sensor support while in FIG. 5B the fetus's head H contacts the extractor's sensor and pushes the sensor support into the extractor cup. FIG. 5C is an expanded view of the sensor, sensor housing, sensor support and connecting wires thereto shown in FIG. 5B while FIG. 5D is a top view of the extractor in FIGS. 5A-5C. FIGS. 5E and 5F are two views of the sensor, sensor housing, and connecting wires thereto.

Vacuum assisted delivery extractor 200 includes a plastic cup 212 having a sensor support 217 positionable within it. The sensor support 217 serves both as the fetal head support discussed in previous embodiments and as a support for a sensor as discussed below. In previous embodiments, the support is typically formed of a flexible plastic whereas in the present embodiment it is made of a rigid plastic. In this embodiment as in previous embodiments, the fetus's head H contacts sensor 218 and pushes against sensor support 217 so that support 217 moves further into cup 212 as in FIG. 5B. Sensor 218 may be denoted herein as a direct wave (DW) sensor receiving physiologically generated electrical waves directly through the skull of the fetus.

Cup 212 is attached to a tubular stem 214, often, but not necessarily, integrally molded to it. Cup 212 and sensor support 217 are typically formed of rigid or semi-rigid plastics, such as, for example, high density polyethylene (HDPE), polypropylene, and acrylonitrile-butadiene-styrene (ABS).

Stem 214 may be in mechanical connection with plastic tubing which extends in the direction of end A. End A is ultimately coupled to a vacuum suction source (not shown). End A of tubular stem 214 may be adapted to be attached to any of many commercially available vacuum suction sources in any of many different ways known to those skilled in the art. Typical commercially available vacuum suction sources that may be used have been discussed herein above in conjunction with previous embodiments.

A pair of isolated electrically conductive wires 220, also often shown separately and designated as wires 220A and 220B, attachable to any of many commercially available monitoring devices, such as fetal heart rate (FHR), fetal pulse rate (FPR), and fetal ECG monitoring devices (not shown) are positioned inside the hollow of tubular stem 214. At a point (not shown) of stem 214 there may be a hermetic seal (not shown) which allows wires 220 to exit the hollow of tubular stem 214 without adversely affecting the vacuum suction applied during a delivery.

The electrically conductive wires, such as wires 220, are generally coated with a non-conductive plastic. Such plastics include, but are not limited to, Teflon® and polyvinyl chloride (PVC).

Isolated electrically conductive wires 220 are brought through the hollow of tubular stem 214 up to plastic tube 213. Tube 213, extending substantially transversally from support 217, may be in mechanical connection or integrally formed with sensor support 217. A first one of wires 220 is then brought to sensor housing 233, the latter typically formed of a rigid plastic. Sensor 218 may be permanently affixed on or in sensor housing 233 and soldered to the first one of the pair of wires 220.

The second wire of the pair of wires 220 passes through tubular stem 214 and enters the inside of cap 212 extending to the rim 249 of cup 212. The wire is in electrical contact with an electrically conductive sensor 250 (best seen in FIGS. 5A, 5B and 5D). The portion of the wire running through the inside of cap 212 to its rim 249 may also be denoted herein as wire 252.

Sensor 218 positioned substantially centrally in cup 212 on floating flexible support housing 233 receives physiologically generated electrical waves directly through the fetal skull. Typically, but without intending to limit the invention, these electrical waves are electrocardiographic (ECG) waves. Sensor 250 positioned on rim 249 of cup 212 receives surface ECG waves that are produced by the heart of the fetus and which travel outside of, and along, the wet fetal body to the head region where they are received by sensor 250.

Sensor 250 may also be denoted herein as a surface wave (SW) sensor receiving electrical waves produced by the fetus that, as noted above, emerge from and travel outside, but generally adjacent to, the fetus. The applicant has noted that use of a sensor to detect surface waves (SW) generated by electrophysiological activity, such as ECG activity, in the fetus improves the reliability of readings of a FSE device. The use of a second electrical sensor 250, the SW sensor, in addition to the first electrical sensor 218, the DW sensor, increases the strength of the signals of a fetal electrocardiogram. Sensor 250 and sensor 218 are both noninvasive with respect to the fetal head. Both sensors are made of electrically conductive metals, such as but without intending to limit the invention, stainless steel. The sensors may be connected to wires 220 by any method known to persons skilled in the art.

Sensor housing 233 is typically recessed within sensor support 217 forming a substantially flat surface therewith, best seen in FIG. 5C.

External to tube 213 and substantially coaxial with it is a spring 215 whose purpose is to provide mechanical support to, and proper positioning of, rigid tube 213 so that it sits substantially collinear with tubular stem 214. The extended and compressed states of spring 215 are shown in FIGS. 5A and 5B respectively.

Sensor 218 may be constructed to be flexible and resilient. Sensor 218 inter alia may have resilient helical (spiral), double helical, coiled and clip shapes. It should readily be understood by those skilled in the art that sensor 218 may have shapes other than those listed above. The shapes listed above are to be considered exemplary only and not limiting. In general, the shape of the sensor may be any shape that allows for good electrical contact with the scalp of the fetus without penetrating the scalp.

Sensor 218 is positioned in sensor housing 233 which is situated in sensor support 217. Cup 212 is typically formed to have a substantially concave profile while sensor support 217 is formed to have substantially flat profile. The concavity of cup 212 allows for the fetal crown to at least partially fit into cup 212. Sensor support 217 is translationally movable within cup 212 in the general direction of end A of tubular stem 214. As indicated in the Figures, sensor 218 is positioned so that it is located generally in the center of support 217 and cup 212, substantially along an axis running through tube 213 and the center of sensor support 217. This is similar to the positioning of sensor 18 in previous embodiments.

When a vacuum suction source (not shown) is attached to end A of tubular stem 214 and activated, the fetal head contacts sensor support 217 and compresses mechanical spring 215. This also pushes sensor support 217 further into cup 212 ensuring good contact between sensor 218 and the fetal scalp. This allows electrical measurements to be made and signals to be sent to the at least one monitor.

Mechanical spring 215 shown in FIGS. 5A-5C allows sensor support 217 to be depressed in the direction of tubular stem 214 as shown in FIG. 5B. This is functionally similar to fetal head support 16 (FIGS. 1A-2B) which because of its inherent flexibility and resiliency is compressible within cup 12 (FIGS. 1A-2B) when the head of the fetus is drawn into cup 12 by the vacuum.

As in previous embodiments, extractor 200 includes a finger grip 228 which is attached to tubular stem 214. Finger grip 228 is used to position extractor 200, as required, during delivery.

Extractor 200 allows medical personnel attending the delivery to monitor the heart beat or pulse or ECG of the fetus on the at least one external monitor (not shown) in electrical or other communication with extractor 200. A typical, but non-limiting, monitor as well as typical, but non-limiting, connectors, adaptors, cables and transducers for use in joining the above monitor to wires 220 extending from sensor 218 have been described herein above in conjunction with previous embodiments.

The electronics required to integrate the sensors and monitors is well known to persons skilled in the art. Such integration, for example, is regularly found in systems employing currently available FSEs with their associated monitors or ECG sensors with their associated monitors. Accordingly, the electronics interface necessary between the at least one monitor used with a vacuum assisted delivery extractor 200 and its associated sensor(s) can readily be developed by persons skilled in the art. Only minor and inconsequential modifications may be required.

Similarly, the sensors for use in FHR and FPR monitoring according to the present invention may be similar in construction to currently commercially available FSE's. Little or no modification would be required of their electronics. Typical sensors have been described herein above in conjunction with previous embodiments.

As in previous embodiments, extractor 200 may be equipped with an emergency suction reduction means (not shown) which can be constructed and operated as described herein above.

While what has been described above discusses the conductive isolated wires as being conducted to the outside of stem 214 of extractor 200 through a hermetic seal (not shown) to one or more monitors (also not shown), wires 220 can conduct signals to a wireless transmitter which can wirelessly communicate with a wireless receiver in electrical communication with one or more monitors. The wireless transmitter (not shown) may be positioned at various sites in extractor 200, for example, inside the hollow of tubular stem 214 or inside finger grip 228.

As in previous embodiments, it is envisioned that the embodiment shown in FIGS. 5A-5F will allow for a warning to be emitted if sensor 218 provides a reading/signal above or below a predetermined value. The warning will indicate to the doctor, midwife or other health professional, that the fetus may be in distress or that a possible dangerous operating condition has developed.

In the embodiment discussed in conjunction with FIGS. 5A-5F, only a single DW sensor 218 is shown and discussed. In other embodiments of the present invention there may be more than a single DW sensor positioned in cup 212 and more than a single measurement concurrently. The plurality of sensors may be of the same type measuring the same physiological indicator or they may be of different types measuring different types of physiological indicators. When a plurality of sensors is used and multiple measurements or multiple types of measurements are made, they may be made concurrently or separately and in series.

In conjunction with FIGS. 5A-5F, only a single SW sensor 250 is shown and discussed. In other embodiments, a plurality of SW sensors 250 may be disposed typically, but without intending to limit the invention, more or less equidistantly around the rim 249 of cup 212.

In the embodiment discussed in conjunction with FIGS. 5A-5F only a single pair of wires is discussed. The present invention also contemplates the possibility that multiple pairs of wires may be connected to a sensor or multiple sensors providing input to multiple monitors, including multiple different types of monitors.

In the embodiment illustrated in FIGS. 5A-5F discussed above, extractor 200 is typically formed of plastic materials except for sensor 218 and wires 220. Sensor 218 being an electrical sensor, and wires 220 connected to it are formed of electrically conductive material. Typical plastics which may be used for forming the remainder of the extractor, and individual pieces thereof when the extractor is not integrally molded, are high density polyethylene (HDPE), polypropylene, acrylonitrile-butadiene-styrene (ABS) and the like.

When multiple plastic pieces are required, such as when joining the cup to the tubular stem 214 or the tubular stem 214 to the vacuum suction source, the joins are hermetic to ensure production of sufficient suction to assist in delivering the fetus.

Sensor 218 of the present embodiment is an active noninvasive sensor, which when used, need only contact the scalp of the fetus. This prevents infections of the scalp as often occurs with other electrodes presently known.

As with previous embodiments, the present embodiments are envisioned as allowing easy placement for occiput posterior or occiput transverse births.

As in previous embodiments, the embodiment shown in FIGS. 5A-5F is also envisioned as a single-use device. Similarly, as in previous embodiments, the extractor may be constructed to allow for use with a standard diameter cup or with cups of different sizes.

Reference is now made to FIGS. 5G-5J where yet another direct wave sensor configuration is shown. Elements found in FIGS. 5G-5J are numbered as in FIGS. 5A-5F if they are present in the latter. FIGS. 5G and 5H are side views of this second direct wave sensor configuration, showing a conducting plate 262 positioned on rigid support 217. The latter is typically formed of a rigid plastic, which supports conducting plate 262 which is in electrical contact with direct wave (DW) sensor 266. Except for DW sensor 266 positioned in hollow tube 263 of conducting plate 262, almost all the elements are constructed and operative substantially as described above in conjunction with FIGS. 5A-5F. The discussion of these similar elements will therefore not be repeated.

Hollow tube 263, typically formed of a conductive material, extends substantially transversally from conducting plate 262 and may be formed integrally therewith. DW sensor 266 is positioned within tube 263 with a wire 220 in contact with, and extending from, sensor 266 into tube stem 214 as in the previous embodiment. Conducting plate 262 is positioned to sit on rigid plastic support 217 which when pressed upon by the head of the fetus descends into cup 212 in the direction of tube stem 214. Conducting plate 262 and hollow tube 263 are formed of an electrically conductive material, typically but without being limiting, stainless steel. Sensor 266 functions as wire sensor 218 in FIGS. 5A-5F.

External to tube 263 and substantially coaxial with it is a spring 215 whose purpose is to provide mechanical support to, and provide proper positioning of, rigid tube 263 so that it sits substantially coaxially with tubular stem 214. The extended and compressed states of spring 215 are substantially the same as shown in FIGS. 5A and 5B respectively.

Another aspect of this embodiment shown in FIG. 5G and not shown elsewhere are wires 220 as they exit from tube stem 214 through a hermetically sealed connector 223. The wires 220 then extend to and connect with a leg plate connector 270 which connects to a leg plate (not shown) worn by the mother during the birthing process. The leg plate allows the monitor reading the fetus's physiologically generated electrical activity to remove (neutralize) background signals arising from the mother's similar electrophysiological activity, typically cardiovascular activity, so that the fetus's readings are more accurate. Conductor 221 connects stem 214 to a vacuum source (not shown).

It should readily be understood by persons skilled in the art that conducting wires 220 of the device shown in FIG. 5A-5F may also be arranged to connect to a maternal leg plate. Such leg plates and leg plate connectors are known in prior art, for example the leg plate and leg plate connectors described in U.S. Pat. No. 5,062,426. The nature and construction of hermetically sealed connector 223 are also well-known to persons skilled in the art.

Rigid plastic support 217 serves both as the fetal head support discussed in previous embodiments and as a support for a conducting plate as described above and shown in the Figures. In some previous embodiments, support 217 may be formed of a flexible plastic whereas in the present embodiment it is made of a rigid plastic. In the present embodiment, the fetus's head H contacts conducting plate 262 pushes against it and rigid plastic support 217 so that support 217 and plate 262 move further into cup 212 as in FIG. 5B.

As may be readily understood by persons skilled in the art, the remainder of the device in FIGS. 5G-5I is constructed and operated as described in conjunction with the embodiment of FIGS. 5A-5F. As readily apparent to persons skilled in the art, the discussions above relating to the embodiment of FIGS. 5A-5F can be applied mutatis mutandis to the embodiment shown in, and discussed in conjunction with, FIGS. 5G and 5I.

FIG. 5I shows the same embodiment as in FIGS. 5G and 5H but a layer 218 is positioned above rigid plastic support 217 and around conducting plate 262. Layer 218 may be formed as an adhesive label or as an adhesive material layer. The adhesive material may be a solid or a gel adhesive and positioned to be exposed to the head of the fetus. The adhesive increases the adhesion of electrically conducting plate 262 to the head, especially to the hair, preventing slippage and disconnection therefrom. Use of adhesive material allows for the adherence of conducting plate 262 at pressures of 0-200 mm Hg without disconnecting from the head of the fetus.

It is contemplated that the embodiments of FIGS. 5A-5I can be used for general monitoring of a fetus even when vacuum assisted delivery (VAD) of the fetus is not required and other birthing procedures are expected to be used. For such situations the diameter of cup 212 may be decreased and a lower vacuum may be used, the vacuum being sufficient for attaching cup 212 to the fetal scalp and contacting the sensor(s) thereto. The cup my be reduced from a cup having a diameter of 50-60 mm to one having a diameter of 20-25 mm and from a vacuum as high as 600 mm Hg to a vacuum in the range of approximately 0-200 mm Hg. Measurements are made with the fetus still inside the mother.

It is also contemplated that the embodiments of FIGS. 5A-5I may be used under non-birthing conditions. Under such conditions, the device is not used as a vacuum extractor having a fetal scalp electrode (FSE). Rather it is used as an electrophysiological monitor of newborns, children or even adults. In such situations, the electrode is operated under suction and contacted to the skin or to a specific body part of the patient and an electrophysiological activity measured. The skin and the body part being contacted should be wet.

FIG. 5J, to which reference is now made, indicates a non-limiting arrangement for positioning SW sensor 250 on the rim of cup 212 (not shown). Element 253 represents a hook positioned over the rim of cup 212 (not shown) which holds SW sensor 250 to the rim. It should readily be appreciated by persons skilled in the art that other methods for attachment of sensor 250 may also be used. The arrangement in FIG. 5J is usable both with the embodiment in FIGS. 5A-5F and the embodiment in FIGS. 5G-5I.

The present invention also provides a method for monitoring the well being of a fetus during a vacuum assisted delivery. The method includes the followings steps:
placing a vacuum assisted delivery extractor on the head of a fetus, the extractor having sensors for sensing electrical signals;
activating a vacuum suction source; and
monitoring the well-being of the fetus by detecting and processing electrical signals of both direct waves and surface waves generated by an electrophysiological event in the fetus.

In this method, a first sensor detects direct waves resulting from an electrophysiological event in the fetus. Direct waves refer to waves that move through the body of the fetus from its point of origin, and are detected by the first sensor as they exit from the body via the skull of the fetus. A second sensor detects surface waves of the electrophysiological event. Surface waves are waves generated within the body of the fetus and which emerge therefrom. After emerging from the body they may travel alongside the body of the fetus within the wet medium in which the fetus resides. They are detected as they arrive at the second sensor positioned at the head of the fetus.

The signals from the direct and surface waves, DW and SW respectively, when added together produce a stronger, clearer signal than if only a single signal were detected. In some embodiments, the joint signal produced by adding the DW and SW signals may have the signal from the mother, as detected by a maternal leg plate, subtracted or otherwise eliminated from the combined DW and SW signal output being processed.

In another embodiment of the method, the individual DW and SW signals can have the signal from the mother, as detected by a maternal leg plate, subtracted or otherwise eliminated from the individual DW and SW signals detected. The stronger and/or clearer signal, that is the DW and SW signal which is stronger and/or clearer after being cleansed of its maternal component, may then be delivered to the monitor for monitoring the well being of the fetus.

In other embodiments, the signal selected for processing may be the combined DW and SW signal or the stronger or clearer of the DW or SW signal, in both cases without having the maternal signal subtracted or otherwise eliminated from the signal(s) of the fetus which are processed.

In the embodiments associated with FIGS. 5A-5I, the terms “electrophysiological event”, “electrophysiological activity” or other such similar terms, typically, but without intending to restrict the invention, refer to events or activities that generate ECG waves.

It is anticipated that the present invention will be widely used by physicians and hospitals as a means of practicing “defensive” medicine thereby contributing to a reduction in malpractice insurance.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. In addition, citation or identification of any reference in this application shall not be construed as an admission that such reference is available as prior art to the present invention. Therefore, it will be appreciated by persons skilled in the art that the present invention is not limited by what has been particularly shown and described herein above. Rather, the scope of the invention is defined by the claims that follow.

What is being claimed is:

1. A delivery extractor for use in a vacuum assisted delivery with a vacuum suction source and at least one monitor for monitoring at least one physiological indicator of the well being of a fetus, said extractor comprising:
a cup-shaped element having a rim;
a tubular stem having a first end and a second end, said first end joined to said cup-shaped element and said second end in pneumatic communication with the vacuum suction source; and
at least one noninvasive first sensor supported within a sensor support, said support depressible within said cup-shaped element and movable therein toward said tubular stem, and at least one noninvasive second sensor positioned on said rim of said cup-shaped element,
wherein said at least one first sensor is operative to detect electrical signals of direct waves arising from an electrophysiological event and said at least one second sensor is operative to detect electrical signals of surface waves arising from the electrophysiological event.

2. A delivery extractor according to claim 1, wherein said at least one first sensor is situated generally in the center of said cup-shaped element so as to be in continuous contact with the scalp of a fetus when the head of the fetus is fully engaged with said cup-shaped element,
and wherein said at least one first and second sensors are in electrical communication with the at least one monitor.

3. A delivery extractor according to claim 1, wherein said at least one second sensor is a plurality of second sensors positioned along said rim of said cup-shaped element.

4. A delivery extractor according to claim 1, wherein said at least one second sensor is positioned on a flexible support adjacent to said rim of said cup-shaped element and depressible therein.

5. A delivery extractor according to claim 1, further including a spring and a sensor housing, said sensor housing being recessed in said sensor support with said at least one first sensor positioned in said sensor housing and protruding therefrom, and said sensor support being in operative connection with the spring so that said sensor support may be depressed within said cup-shaped element.

6. A delivery extractor according to claim 1, wherein said at least one first sensor and said at least one second sensor monitor at least one of the following physiological indicators of the well being of the fetus: fetal heart rate (FHR), fetal pulse rate (FPR), and fetal ECG.

7. A delivery extractor according to claim 1, wherein said first and second sensors are in electrical communication with a wireless transmitter, said wireless transmitter transmitting signals to a wireless receiver in electrical communication with the at least one monitor.

8. A delivery extractor according to claim 1, wherein said sensor support has a conductive plate positioned thereon, said plate having a tube integrally formed therewith extending substantially transversally from said plate and said at least one first sensor is configured and sized to be positioned and held within said tube.

9. A system for monitoring the condition of a fetus during a vacuum assisted delivery, said system comprising:
a delivery extractor comprising:
a cup-shaped element having a rim;
a tubular stem having a first end and a second end, said first end joined to said cup-shaped element; and
at least one noninvasive first sensor supported within a sensor support, said support depressible within said cup-shaped element and movable therein toward said tubular stem, and at least one noninvasive second sensor positioned on said rim of said cup-shaped element,
wherein said at least one first sensor is operative to detect electrical signals of direct waves arising from an electrophysiological event and said at least one second sensor being operative to detect electrical signals of surface waves arising from the electrophysiological event;
a vacuum suction source removably connectable with said second end of said tubular stem and in pneumatic communication with said cup-shaped element; and
at least one monitor in communication with said at least one first sensor and said at least one second sensor receiving signals therefrom, said at least one monitor monitoring at least one physiological indicator of the well-being of the fetus.

10. A system according to claim 9, wherein said at least one first sensor is situated generally in the center of said cup-shaped element so as to be in continuous contact with the scalp of a fetus when the head of the fetus is fully engaged with said cup-shaped element, said at least one first sensor and said at least one second sensor are in electrical communication with said at least one monitor.

11. A system according to claim 9, further including a spring and a sensor housing, said sensor housing being recessed in said sensor support, wherein said at least one first sensor is positioned in said sensor housing and protruding therefrom, and said sensor support is in operative connection with the spring so that said sensor support may be depressed within said cup-shaped element.

12. A system according to claim 9, wherein said stem is equipped with an emergency suction reduction means which can be activated to interrupt the suction produced by said vacuum suction source when said at least one first sensor and said at least one second sensor indicate that the fetus may be in distress or when a physician has otherwise determined that the vacuum delivery procedure must be aborted.

13. A system according to claim 9, wherein said stem is equipped with a vacuum modulating means operative to modulate the vacuum produced by said vacuum suction source when a physician has determined that the vacuum strength is to be increased or decreased during the delivery.

14. A system according to claim 9, further including a wireless transmitter in electrical communication with said at least one first sensor and at least one second sensor receiving electrical signals therefrom so as to transmit signals to a wireless receiver in electrical communication with said at least one monitor.

15. A system according to claim 9, wherein said at least one monitor further includes a controller that disconnects said vacuum suction source when said controller detects that the measured physiological indicator of well being is greater or less than predetermined maximum and minimum values for that indicator.

16. A system according to claim 9, wherein said vacuum suction source includes a controller which is operative to disconnect said vacuum suction source when the suction produced by said source exceeds a predetermined value for the maximum suction to be applied or when the total duration during which maximum suction has been applied is greater than a predetermined value for the total duration for which the maximum suction is to be applied.

17. A system according to claim 9, wherein said at least one second sensor is a plurality of second sensors positioned along said rim of said cup-shaped element.

18. A system according to claim 9, wherein said at least one second sensor is positioned on a flexible support adjacent to said rim of said cup-shaped element and depressible into said cup-shaped element.

19. A system according to claim 9, wherein said sensor support has a conductive plate positioned thereon and has a tube integrally formed therewith extending substantially transversally from said plate and said at least one first sensor is configured and sized to be positioned and held within said tube.

20. A system according to claim 9, further including a maternal leg plate in electrical communication with said monitor, the maternal signals detected by said leg plate being subtracted, or otherwise eliminated, from the output data obtained from the detected direct and surface wave signals of the fetus.

21. A method for monitoring the well being of a fetus during a vacuum assisted delivery, the method including the followings steps:
placing a vacuum assisted delivery extractor on the head of a fetus, the extractor having at least two sensors for sensing electrical signals;
activating a vacuum suction source; and
monitoring the well-being of the fetus by detecting and processing signals from both direct waves and surface waves generated by an electrophysiological event in the fetus

22. A method according to claim 21, wherein said step of monitoring further includes a step of combining the direct wave and surface wave signals detected by the at least two sensors.

23. A method according to claim 21, wherein said step of monitoring further includes a step of subtracting out, or otherwise eliminating, the maternal signal detected by a maternal leg plate from the output data obtained from the detected direct and surface waves signals of the fetus.

24. A method according to claim 21, wherein said step of monitoring further includes a step of selecting the cleaner, stronger signal from between the direct and surface wave signals of the fetus after the direct and surface wave signals have had any maternal signal subtracted out, or otherwise eliminated.

25. A method according to claim 21, wherein said step of activating includes allowing the vacuum suction to reach a first value during the mother's contractions and then allowing the vacuum suction to reach a second value between contractions, where the second value is less than the first value.

26. A method according to claim 21, further including the step of aborting said step of activating a suction source when at least one of the following conditions is indicated: said step of monitoring indicates that the fetus is in distress; said step of monitoring indicates that the maximum suction being applied exceeds a predetermined value for the maximum suction to be applied; and said step of monitoring indicates that the total duration at which the maximum suction has been applied exceeds a predetermined value for the maximum duration.

27. A method according to claim 21, further including the step of modulating the strength of the vacuum produced by the vacuum suction source when at least one of the following conditions is indicated: said step of monitoring indicates that the fetus is in distress; said step of monitoring indicates that the maximum suction being applied exceeds a predetermined value for the maximum suction to be applied; said step of monitoring indicates that the total duration at which the maximum suction has been applied exceeds a predetermined value for the maximum duration; and the physician has determined that the vacuum suction strength is to be increased or decreased.

28. A method according to claim 21 wherein said step of placing a vacuum assisted delivery extractor on the head of a fetus, includes positioning against the scalp of the fetus at least two noninvasive sensors for monitoring one or more physiological indicators of the well-being of the fetus, at least one sensor detecting electrical signals of direct waves arising from an electrophysiological event and passing through the fetal skull to the sensor and at least one sensor detecting electrical signals of surface waves arising from the electrophysiological event passing through a wet environment surrounding the fetus, the waves related to the physiological indicators of well being.

29. A method for monitoring according to claim 21, wherein said extractor is used as an instrument for internal monitoring of the well being of a fetus when a vacuum-assisted birth is not contemplated and said step of activating activates the suction source at lower pressures than the pressure levels used when a vacuum-assisted birth is contemplated.

30. A method for monitoring according to claim 21, wherein said extractor is used as an external monitoring instrument of the physiological indicators of well being of a patient when the extractor is suctionally attached to the patient's skin, and during said step of activating the vacuum is activated at lower pressures than pressure levels used when a vacuum-assisted birth is contemplated but still sufficient to ensure that the cup-shaped element is attached to the skin of the patient.

31. A method for monitoring according to claim 30, wherein when the skin is dry, the method further includes a step of wetting the skin.
